(19) United States
(12) Patent Application Publication
(10) Pub. No.: US 2021/0062146 A1
(43) Pub. Date: Mar. 4, 2021

(54) OPTIMIZED IN VITRO CELLULAR SYSTEM FOR BBB PERMEABILITY LINKED NEUROACTIVITY SCREENING

(71) Applicant: Purdue Research Foundation, West Lafayette, IN (US)

(72) Inventors: Gregory T. Knipp, West Lafayette, IN (US); Kelsey E. Lubin, Indianapolis, IN (US)

(73) Assignee: Purdue Research Foundation, West Lafayette, IN (US)

(21) Appl. No.: 17/004,080

(22) Filed: Aug. 27, 2020

Related U.S. Application Data
(60) Provisional application No. 62/894,079, filed on Aug. 30, 2019.
(60) Provisional application No. 62/893,522, filed on Aug. 29, 2019.

(51) Int. Cl. C12N 5/079; C12N 5/071

(52) U.S. Cl. CPC C12N 5/0622; C12N 5/0697; C12N 2533/30; C12N 2502/28; C12N 2502/086

(57) ABSTRACT

The present disclosure generally relates to a process to prepare a cell culture system that mimics the structure of Blood Brain Barrier (BBB) and are useful to study the functions thereof. In particular, the present invention relates to a direct-contact triculture systems prepared by plating BMECs on a pre-formed lawn of coculture of astrocytes and pericytes on the apical surface of a culture-chamber to achieve a truly direct contact triculture model for BBB. The cell culture systems disclosed herein are optimized using a method called Design of Experiments (DOE) useful for studying the functions of the Blood Brain Barrier and predicting the efficacy and/or potential toxicity of a drug candidate.

### CROSS REFERENCE TO RELATED APPLICATION

This present patent application relates to and claims the priority under 35 U.S.C. § 119(e) to U.S. Provisional Patent Application Ser. No. 62/893,522 filed on Aug. 29, 2019, and to U.S. Provisional Patent Application Ser. No. 62/894,079 filed on Aug. 30, 2019, the contents of which are hereby incorporated by reference in their entirety into the present disclosure.

### TECHNICAL FIELD

The present disclosure generally relates to a process to prepare a multiple-cellular, direct contact layered in vitro culture system that mimics the structure of the in vivo Blood Brain Barrier (BBB) and are useful to study the functions thereof. In particular, the present invention relates to a multi-cellular neurovascular unit (NUV) comprising astrocytes, pericytes and human brain derived endothelial cell line cultured together on the apical surface of a filter support with a neuron cell line cultured below to achieve a truly direct contact triculture model for BBB with the ability to assess permeability-linked neuronal response in the NVU. The cell culture systems disclosed herein are optimized using a method called Design of Experiments (DOE) useful for optimizing a cell culture model to investigate the functions of the Blood Brain Barrier and neurons when grown together that aims to provide and in vitro means to predicting the efficacy and/or potential toxicity of a drug candidate. The cell culture systems and methods of manufacture are within the scope of this disclosure.

### BACKGROUND

This section introduces aspects that may help facilitate a better understanding of the disclosure. Accordingly, these statements are to be read in this light and are not to be understood as admissions about what is or is not prior art.

The Blood Brain Barrier (BBB) was first reported in the late 1800s by Paul Erhlich, when he first noticed that certain staining dyes (hydrophilic dyes) could stain most body organs except the brain. Further studies by his student Edwin Goldmann proved that there was a barrier between the blood and the brain, which could not allow the passage of hydrophilic dyes (Ribatti, D., J. Anat 2006, 208(2): 139-152). After many years of research, it has been now established that brain capillaries are functionally and physiologically different from capillaries found in other parts of the central nervous system (CNS) and the body.(Banks 2016) A particularly important attribute of the BBB is that junctional complexes lining the paracellular space between the brain microvessel endothelial cells (BMECs) that form the capillary are comprised of distinct protein complexes that form a highly restrictive sieving barrier to the movement of most small and large hydrophilic molecules (Myers, M.G. Ann Neurol. 2013, 1(5), 409-417).

In addition, brain capillaries have little or no pinocytosis, possess a higher number of mitochondria (indicating higher metabolic requirements) and have a unique environment in which BMECs are almost completely surrounded by astrocytic end feet and pericytes (Turowski and Kenny, Front Neurosci. 2015, 9,156). Therefore, the uniqueness of the BMECs and their environment result in the formation of a physiologically dynamic barrier that restricts the transport of most therapeutic drugs designed to treat neurological disorders (Pardridge, W. Expert Opin Drug Deliv, 2016, 13(7):963-975). A major challenge exists in modeling BBB permeation using simple and robust in vitro techniques.

Current in vitro techniques to model the BBB involve seeding BMECs on membranous filter support in as either a monolayer or in configuration with astrocytes and/or pericytes in the bottom chamber of TRANSWELL® system (Banerjee, J. et al., Drug Discov Today, 2016, 21(9), 1367-1386). It is important to note that the BMECs can be derived from several different species including rats, bovine, and porcine sources (Helms, H. et al., J. Cerebral Blood Flow Metabolism, 2016, 36(5): 862-890). They can also be primary or transformed cells, which may further confound extrapolation to human BBB penetration (Syvanen, Lindhe et al. Drug Metab Dispos, 2009, 37(3): 635-643). More recently, induced pluripotent stem cells have been utilized to derive BBB cell phenotypes and culture for in vitro testing (Lippmann ES, et al., Fluids Barriers CNS. 2013, 10(1):2). In a monoculture configuration, the BMECs lack a physiologically relevant environment without biochemical signaling or physical interaction with supporting BBB cells such as astrocytes and pericytes.

In many cases, an effort to introduce the BBB relevant environment in the in vitro models has been conducted by seeding either astrocytes or pericytes directly under the membranous filter support. However, this approach does not allow optimal (and physiologically representative) direct interactions between astrocytes, pericytes and endothelial cells as observed at the in vivo-BBB. The TRANSWELL® filter support provides a significant limitation to the achievement of physical coverage and cell-cell connections formed, especially, between astrocytes and/or pericytes with the BMECs at the BBB in vivo when cultured in an indirect configuration. Furthermore, conventional indirect triculture, and even coculture, systems have been developed where either the astrocytes or pericytes are cultured on the bottom of the filter or alternatively pericytes or astrocytes, respectively, are cultured on the bottom of the basal chamber (Hatherell, Couraud et al. J Neurosci Methods, 2011, 199(2):223-229). The filter represents a 10 uM, semiporous physical barrier between the cell types, whereas in vivo the extracellular matrix represents is an approximate barrier distance of 20 nM. While these models have provided significant reduction in permeability of paracellular markers, they still lack the extent of restriction and a physiologically representative BBB-configuration as found in vivo.

The restrictive properties of the Blood Brain Barrier (BBB) are largely influenced by the presence of supporting cells (astrocytes and pericytes) of the neurovascular unit (NVU), which underlie the brain microvessel endothelial cells (BMEC) in close proximity. In vivo relevant direct contact between astrocytes, pericytes, and BMECS to our knowledge has not been established in conventional TRANSWELL® based in vitro screening models of the BBB.

We have previously established a direct contact triculture model to mimic the in vivo NVU by developing direct layered contact of astrocytes, pericytes, and BMECs layered on the apical surface of a permeable filter support with hCMEC/D3 cells (Knipp, et al., U.S. Ser. No. 15/697,699, filed Sep. 7, 2017). Due to some concerns regarding the loss of phenotypic similarity with increasing passages, the hCMEC/D3 cell line was replaced. Here we describe the utilization of primary human astrocytes and pericytes cultured with the human brain endothelial cells (HBEC-5i), where culturing conditions were optimized using a design of experiments (DOE) based approach to arrive at optimized conditions using the multi-factor cell based system. We have demonstrated that a DOE approach is a useful tool to expedite optimization of biologically based systems and facilitates understanding of factor interactions in these models. The direct contact model has been shown to provide increased NVU-like restricted permeation comparative to HBEC-5i monoculture and direct contact coculture models, suggesting that the presence of both astrocytes and pericytes in physiologically relevant contact with the endothelium further enhances the restrictive NVU phenotype.

### BRIEF DESCRIPTION OF THE DRAWINGS

The disclosed embodiments and other features, advantages, and disclosures contained herein, and the matter of attaining them, will become apparent and the present disclosure will be better understood by reference to the following description of various exemplary embodiments of the present disclosure taken in conjunction with the accompanying drawings, wherein:

FIG. 1A shows a cross section depicting the neurovascular unit (NVU) with the endothelium (BMECs) lining the capillary, pericytes embedded within the basal lamina, astrocytes having nearly full coverage of the BMECs and surrounding pericytes, and neurons in close contact with the astrocytes. FIG. 1B depicts the direct contact triculture model on the apical surface of a TRANSWELL® filter support mimicking the in vivo NVU. Astrocytes are seeded first on the filter, followed by pericytes, then BMECs to generate a fully apical, direct contact triculture model.

FIG. 2 shows Pₐₚₚ and P_{eff} of 4 kD FITC-Dextran across different direct contact triculture conditions of DOEₚ. All conditions were performed as n=1. Condition 13 was compromised and permeability was not performed, data point was excluded from statistical analysis.

FIG. 3 shows P_{eff} of 4 kD FITC-Dextran for DOEₚ separated by factor and further by day of study showing relative trends of factor levels at increasing length of culture. All conditions are represented by single data points across the graph, n=1.

FIG. 4 depicts JMP 13.2 Prediction Profiler generated based on maximizing desirability for P_{eff} based on DOEₚ. Optimal plating conditions 20,000 cells/cm² astrocytes and pericytes, 80,000 cells/cm² HBEC-5i, MATRIGEL®, and 9 days of endothelial growth. Predicted P_{eff} of 2.4×10⁻⁶ cm/sec for optimal conditions.

FIG. 5 shows JMP 13.2 Prediction Profiler generated based on maximizing desirability for P_{eff} of DOE_{M1}. Optimal medium conditions 15 mM HEPES, 1 mM Ca²⁺, and 10 μM retinoic acid at 9 days of endothelial growth. Predicted P_{eff} of 7.0×10⁻⁶ cm/sec for optimal conditions.

FIG. 6 shows JMP 13.2 Prediction Profiler generated based on maximizing desirability for P_{eff} of DOE_{M2}. Optimal medium conditions 10 μM dexamethasone, 10 μM retinoic acid, 10 mM LiCl, through 7 days of endothelial culture. Predicted P_{eff} of 8.8×10⁻⁶ cm/sec for optimal conditions.

FIG. 7 demonstrates the effective permeability (P_{eff}) of 4 kD FITC-dextran across an HBEC-5i monoculture, pericyte-HBEC-5i direct contact coculture, astrocyte-HBEC-5i direct contact coculture, and optimized direct contact triculture. Statistical analysis was performed with one-way ANOVA and Tukey-Kramer post-hoc test. Error bars represent one standard deviation (n=3). *, p<0.05 and **, p<0.01.

FIG. 8 shows the apparent permeability of radiolabeled paracellular markers [¹⁴C]-sucrose. [¹⁴C]mannitol, [¹⁴C]-inulin, and [¹⁴C]-PEG-4000 across the optimized direct contact triculture. Error bars represent one standard deviation.

FIG. 9 shows the apparent permeability of P-gp substrate rhodamine 123 (R123) in the presence and absence of P-gp inhibitor elacridar across the optimized direct contact triculture. Assays were run in triplicate and subjected to Student's t-test. Significant difference is indicated by *, p<0.05 and **, p<0.01. Error bars represent one standard deviation (n=3).

FIG. 10 shows the apparent permeability of BBB positive (L-histidine, carbamazepine, and rhodamine 123 in the presence of P-gp inhibitor elacridar) and negative (colchicine, rhodamine 123, digoxin, clozapine, and prazosin) permeants across the optimized direct contact triculture. Assays were performed in triplicate. Error bars represent one standard deviation (n=3).

FIG. 11 illustrates a cross sectional depiction of the in vitro neurovascular unit (NVU) with the endothelium (BMECs) lining the capillary, pericytes embedded within the basal lamina, astrocytes having nearly full coverage of the BMECs and surrounding pericytes, and neurons in close contact with the astrocytes.

FIG. 12 shows the apparent permeability (Papp) of a 4 kD FITC-dextran across the direct contact triculture alone (dark grey) and the optimized NVU model (light grey) with neurons in the basolateral chamber. Optimized conditions for the NVU model consists of 25,000 cells/cm² SH-SY5Y neurons introduced to the apical direct contact triculture 3 days post endothelial cell plating. Error bars represent one standard deviation (n=3).

FIG. 13 demonstrates the viability and outgrowth of SH-SY5Y neurons in culture with and without the presence of the direct contact triculture as measured by the Neurite Outgrowth Staining Kit. Viability and outgrowth of SH-SY5Y cells cultured alone were normalized to 100% for comparison. Statistical significance was determined using Student's t-test between the two groups. Significance is labeled by (viability, outgrowth) where * p<0.05, ** p<0.01. Error bars represent one standard deviation (n=5).

FIG. 14 shows the apparent permeability of paracellular marker compounds [¹⁴C]-sucrose and FITC-dextrans of 4 kD, 10 kD, and 40 kD across the optimized NVU model. Error bars represent one standard deviation (n=3).

FIG. 15 depicts the apparent permeability of P-gp substrate rhodamine 123 (R123) in the presence and absence of elacridar, a P-gp inhibitor. Papp was measured across the triculture without neurons (dark grey) and the NVU model (light grey). One way ANOVA with a Tukey-Kramer post-hoc test was used to determine significance between R123 with and without elacridar for the triculture with neurons and without where *p<0.05, **p<0.01, and ***p<0.001. Error bars represent one standard deviation (n=3).

FIG. 16 shows the apparent permeability of BBB positive (grey) and negative (patterned) permeants across the optimized NVU model. Assays were performed in triplicate where error bars represent one standard deviation (n=3−6).

FIG. 17 demonstrates the apparent permeability of high and low permeating marker compounds across the optimized direct contact triculture (dark grey) and the optimized NVU model (light grey). Student's t-test was used to determine statistical significance between Pₐₚₚ of markers across the two models where *p<0.05, **p<0.01, and ***p<0.001. Error bars represent one standard deviation (n=3-6).

FIG. 18 shows the neuroactivity of marker compounds after 3 hour permeability measured by neuronal viability (solid) and outgrowth (checkered) of SH-SY5Y cells represented as percentage of control NVU neuronal cells with vehicle (0.50% DMSO) alone and relative flux of each marker compound (red circle). Initial concentrations of 50 μM were placed in the apical chamber of the NVU model, SH-SY5Y neurons were stained for viability and outgrowth at the end of 3 hours of marker permeation. Viability stain was measured at ex: 483 nm and em: 525 nm and outgrowth stain was measured at ex: 535 nm and em: 590 nm. Markers are ordered based on highest to lowest permeation rate across the NVU model. Statistical significance was determined using a one way ANOVA followed by a Tukey-Kramer post-hoc test where *p<0.05, **p<0.01, and ***p<0.001. Error bars represent one standard deviation (n=4).

FIGS. 19A-19C depict the qualitative fluorescence (top) and bright field (bottom) images of SH-SY5Y neuronal cells after 3 hours of (FIG. 19A) control vehicle (0.50% DMSO), (FIG. 19B) digoxin, and (FIG. 19C) cyclosporin A accumulation after permeation across the apical triculture in the optimized NVU model. Images were obtained using the BioTek Cytation 3 with 20× objective and Green Fluorescent Protein and Texas Red filters for neuronal viability and outgrowth respectively. Scale bars represent 100 μm.

### DETAILED DESCRIPTION

For the purposes of promoting an understanding of the principles of the present disclosure, reference will now be made to the embodiments illustrated in the drawings, and specific language will be used to describe the same. It will nevertheless be understood that no limitation of the scope of this disclosure is thereby intended.

In the present disclosure the term “about” can allow for a degree of variability in a value or range, for example, within 20%, within 10%, within 5%, or within 1% of a stated value or of a stated limit of a range.

In the present disclosure the term “substantial” or “substantially” can allow for a degree of variability in a value or range, for example, within 80%, within 90%, within 95%, or within 99% of a stated value or of a stated limit of a range.

In this document, the terms “a,” “an,” or “the” are used to include one or more than one unless the context clearly dictates otherwise. The term “or” is used to refer to a nonexclusive “or” unless otherwise indicated. In addition, it is to be understood that the phraseology or terminology employed herein, and not otherwise defined, is for the purpose of description only and not of limitation. Any use of section headings is intended to aid reading of the document and is not to be interpreted as limiting. Further, information that is relevant to a section heading may occur within or outside of that particular section. Furthermore, all publications, patents, and patent documents referred to in this document are incorporated by reference herein in their entirety, as though individually incorporated by reference. In the event of inconsistent usages between this document and those documents so incorporated by reference, the usage in the incorporated references should be considered supplementary to that of this document; for irreconcilable inconsistencies, the usage in this document controls.

In some illustrative embodiments, the present disclosure relates to a method to prepare a multiple-cellular cell culture system with a neuronal cell line that is useful for Blood Brain Barrier (BBB) permeability linked neuroactivity evaluation. In particular, the present invention relates to a multi-cellular neurovascular unit (NUV) comprising astrocytes, pericytes and human brain derived endothelial cell line together with a neuron cell line. The cell culture systems and methods of manufacture are within the scope of this disclosure.

In some illustrative embodiments, the present disclosure relates to a method for preparing a multi-cellular neurovascular unit (NUV) for Blood Brain Barrier (BBB) linked neuroactivity evaluation comprising the steps of:
a) preparing a cell culture plate with a first support membrane;
b) seeding a first cell line on said first support membrane and proliferating said first cell line;
c) seeding a second cell line over said first cell line and proliferating said second cell line;
d) coating the lawn of said first and second cell lines with an extracellular matrix (ECM);
e) seeding a third cell line over ECM coated proliferated cell lawn of said first and second cell line and proliferating said third cell line together with said first and second cell lines to afford a triculture system;
f) seeding a neuron cell line or a surrogate on a second support membrane; and
g) placing said first support membrane comprising said triculture system above said neuron cell line or a surrogate into a cell culture chamber to afford an in vitro neurovascular unit (NVU).

In some illustrative embodiments, the present disclosure relates to a method for preparing a multi-cellular neurovascular unit (NUV) for Blood Brain Barrier (BBB) linked neuroactivity evaluation as disclosed herein, wherein said first cell line is astrocytes or other glial cells and said second cell line is pericytes.

In some illustrative embodiments, the present disclosure relates to a method for preparing a multi-cellular neurovascular unit (NUV) for Blood Brain Barrier (BBB) linked neuroactivity evaluation as disclosed herein, wherein said first cell line of astrocytes or other glial cells has a seeding density of about 20,000 cells/cm² and said second cell line of pericytes has a seeding density of about 20,000 cells/cm², optimizable using the method Design of Experiments (DOE) as described in this present disclosure.

In some illustrative embodiments, the present disclosure relates to a method for preparing a multi-cellular neurovascular unit (NUV) for Blood Brain Barrier (BBB) linked neuroactivity evaluation as disclosed herein, wherein said third cell line is proliferative human derived cerebral microvessel endothelial cells (HBEC-5i).

In some illustrative embodiments, the present disclosure relates to a method for preparing a multi-cellular neurovascular unit (NUV) for Blood Brain Barrier (BBB) linked neuroactivity evaluation as disclosed herein, wherein said third cell line is preprogrammed induced pluripotent stem cells.

In some illustrative embodiments, the present disclosure relates to a method for preparing a multi-cellular neurovascular unit (NUV) for Blood Brain Barrier (BBB) linked neuroactivity evaluation as disclosed herein, wherein said third cell line comprises brain microvessel endothelial cells (BMECs) of human or animal origin, primary, immortalized, normal or in a diseased state, or Human Brain Endothelial Cells (HBECs).

In some illustrative embodiments, the present disclosure relates to a method for preparing a multi-cellular neurovascular unit (NUV) for Blood Brain Barrier (BBB) linked neuroactivity evaluation as disclosed herein, wherein said third cell line of brain microvessel endothelial cells (BMECs) of human or animal origin, primary, immortalized, normal or in a diseased state, or Human Brain Endothelial Cells (HBECs) has a seeding density of about 80,000 cells/cm², optimizable using the method Design of Experiments (DOE) as detailed in this present disclosure.

In some illustrative embodiments, the present disclosure relates to a method for preparing a multi-cellular neurovascular unit (NUV) for Blood Brain Barrier (BBB) linked neuroactivity evaluation as disclosed herein, wherein said neuron cells or a surrogate neuronal cells comprises human neuroblastoma cell lines, preprogrammed induced pluripotent stem cells of differing phenotypes, isolated primary human or animal neurons that are obtained from different brain sections, and human or animal neuronal cell lines.

In some illustrative embodiments, the present disclosure relates to a method for preparing a multi-cellular neurovascular unit (NUV) for Blood Brain Barrier (BBB) linked neuroactivity evaluation as disclosed herein, wherein said a neuronal cell line has a seeding density of from about 5,000 to about 500,000 cells/cm², optimizable using the method Design of Experiments (DOE) as described in this present disclosure.

In some illustrative embodiments, the present disclosure relates to a method for preparing a multi-cellular neurovascular unit (NUV) for Blood Brain Barrier (BBB) linked neuroactivity evaluation as disclosed herein, wherein performance of said NVU cell culture system is optimal when proliferated cell lines reach their confluency.

In some other illustrative embodiments, the present disclosure relates to a multi-cellular cell culture system of in vitro neurovascular unit (NVU) for Blood Brain Barrier (BBB) linked neuroactivity evaluation comprising:
a) a cell culture plate with a first support membrane;
b) a triculture system comprising a first cell line, a second cell line and a third cell line, wherein said first cell line and second cell line are seeded on said first support membrane and proliferating before coating the lawn of said first and second cell lines with an extracellular matrix (ECM) and then seeding said third cell line to enable BBB formation;
c) a neuron cell line or a surrogate, wherein said neuron cell line or surrogate is seeded on a second support membrane prior to placing said triculture system atop said neuron cell line or a surrogate to afford an in vitro neurovascular unit (NVU); and
d) said NVU is maintained with complete endothelial medium on the apical side of the filter and complete neuronal medium in the basolateral chamber until being demonstrated as optimal for permeability and neuroactivity assays.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said first cell line is astrocytes or other glial cells and said second cell line is pericytes.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said first cell line of astrocytes or other glial cells has a seeding density of about 20,000 cells/cm² and said second cell line of pericytes has a seeding density of about 20,000 cells/cm², optimizable using the method Design of Experiments (DOE) as detailed in this disclosure.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said third cell line is proliferative human derived cerebral microvessel endothelial cells hCMEC/D3 or preprogrammed induced pluripotent stem cells.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said third cell line is brain microvessel endothelial cells (BMECs) of human or animal origin, primary, immortalized, normal or in a diseased state, or Human Brain Endothelial Cells (HBECs).

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said third cell line of brain microvessel endothelial cells (BMECs) of human or animal origin, primary, immortalized, normal or in a diseased state, derived from induced pluripotent stem cells, or a surrogate of Brain Endothelial Cells (HBECs) has a seeding density of about 80,000 cells/cm², optimizable using the method Design of Experiments (DOE) detailed in this disclosure.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said neuron cell or surrogate comprises human neuroblastoma cell lines, preprogrammed induced pluripotent stem cells of differing phenotypes, isolated primary human or animal consisting of both healthy or diseased state neurons that are obtained from different brain sections, and human or animal neuronal cell lines.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said human neuroblastoma cell line has a seeding density of about 5,000 to about 500,000 cells/cm², optimizable using the method Design of Experiments (DOE) as detailed in this disclosure.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein performance of said NVU cell culture system is at its best when proliferated cells reach their confluency.

In some other illustrative embodiments, the present disclosure relates to a multi-cellular cell culture system of in vitro neurovascular unit (NVU) for Blood Brain Barrier (BBB) linked neuroactivity evaluation as disclosed herein, wherein said proliferated cells reach their confluency in about nine days after seeding of the third cell line.

There is a continuing need for screening models that will facilitate the development of therapeutic agents aimed at mitigating brain disorders, particularly as there is a rapidly increasing prevalence of neurodegenerative and neurodevelopmental diseases. The costs associated with developing neurological therapeutics is significant in a large part due to the high rates of attrition in later stages of development. The implementation of a low cost, predictive, and physiologically relevant in vitro screening model to more rigorously facilitate hit and lead candidate selection providing greater in vivo correlative rank ordering of potential compounds or drug delivery systems for further development is imperative.

Many have theorized that the high rates of attrition are predominantly due to the in ability of drug candidates to cross the Blood Brain Barrier (BBB) (Pardridge W M, Drug Discovery Toady 2002; 7:5-7; Gribkoff V K et al., Neuropharmacology 2017; 120:11-19). The BBB has traditionally been believed to be comprised of brain microvessel endothelial cells (BMECs) that line the capillaries of the brain to maintain a homeostatic environment. The BBB separates the brain parenchyma from the systemic circulation and prevents permeation of potential xenobiotics into the brain milieu (Abbott N J et al., Neurobiol. Dis. 2010; 37:13-25). The BBB endothelium is unique in comparison to the periphery due to the high expression of efflux proteins, drug transporters, metabolizing enzymes, and the presence of restrictive tight junctions. Tight junctions in the brain are formed between adjacent BEMCs by a complex of transmembrane intracellular cleft spanning proteins such as occludin and claudins 3 and 5, which anchor to cytosolic scaffolding proteins supported by the actin cytoskeleton (Bauer H-C et al., Front Neurosci. 2014; 8:392). The presence of restrictive tight junctions limits the permeation of small hydrophilic compounds, forcing compounds to move transcellularly in order to cross the BBB. The high expression levels of non-substrate specific ATP-binding cassette (ABC) transporters such as P-glycoprotein (P-gp) and Breast Cancer Resistance Protein (BCRP) results in a high degree of efflux for molecules that attempt to cross the BBB through the transcellular pathway. The presence of efflux transporters may limit the permeation of potential neurotoxicants, while also presenting a challenge for drug delivery as a number of intended neurotherapeutics tend to be lipophilic, favoring multidrug-resistant isoform efflux (Polli J W et al., Drug Metab Dispos 2009; 37:439-442). Due to their unique presence in the BBB, restrictive tight junctions and functional efflux proteins are key validation characteristics when establishing an in vitro BBB screening model.

The in vivo BBB phenotype is also largely modulated by the presence of supporting cellular and non-cellular components including astrocytes, pericytes, neurons, and the basal lamina. Together, these components make up the neurovascular unit (NVU), which are each essential for the function of the BBB in vivo. Astrocytes fully surround the endothelium and are linked to each other via gap junctions. Single astrocytes have been shown to interact with up to four different neurons and five blood vessels, making them the cellular link between the endothelium and brain parenchyma.¹⁴⁻¹⁶ Astrocytes participate in ion and water regulation due to the localization of these channels in the astrocytic endfeet and has been linked to the expression of basal lamina proteins. Additionally, astrocytes influence BMEC growth, modulation through extracellular signaling, play an important metabolic role, and assist in the functional maintenance through the secretion of soluble factors which have been shown to be essential for NVU homeostasis. Towards the latter point, several in vitro and in vivo studies have demonstrated that changes in BBB integrity may result from a deficiency of certain astrocytic soluble factors (Haseloff R F et al., Cell Mol Neurobiol 2005; 25:25-39; Alvarez J I, et al., GLIA 2013; 61:1939-1958).

Pericytes are found enveloped in the basal lamina of the NVU between the astrocytes and endothelium. However, pericyte distribution is not continuous and in general cover approximately one third of the BMEC basal layer, with higher densities observed regiospecifically within the brain. Pericytes are believed to play a similar role as astrocytes in NVU modulation through the secretion of soluble factors, but are unique in their role in NVU formation and maintenance, specifically during development (Winkler E A et al., Nat Neurosci 2011; 14:1398-1405; Daneman R et al., Nature 2010; 468:562-566). Pericyte-endothelial crosstalk occurs through a number of signal cascades including platelet-derived growth factor B (PDGF-B) and transforming growth factor-β (TGF-β), as well as others. Interactions between the pericytes and endothelium occurs within the basal lamina due to the relative location of embedded pericytes in the shared basement membrane, potentially suggesting that the composition of the extracellular matrix plays a role in BBB development and maintenance. The basal lamina is a non-cellular component of the NVU and is responsible for maintaining integrity of the BBB by anchoring the cellular components. There are a significant number basement membrane proteins that include fibronectin, collagen IV, laminins, and vitronectin that form the matrix which is approximately 20 nm thick in vivo. Given the multiple components that make up the NVU, cellular and non-cellular, we hypothesize that the BBB should be viewed as the NVU as a whole rather than simply the contributions of the BMECs.

In vitro screening models have traditionally been used to evaluate the potential of new chemical entities to cross the BBB, with much of the emphasis of these models being placed on the endothelial cell type. The BMEC used is often primary or immortalized and of animal or human origin, each presenting its own advantages for use in in vitro models (Helms H C et al., J Cereb Blood Flow Metab Off J Int Soc Cereb Blood Flow Metab 2016; 36:862-90; Naik P et al., J Pharm Sci 2012; 101:1337-13354). Although animal sources are typically lower cost, have significantly higher access, and can be easier to isolate, physiological and phenotypic differences between the human and animal NVU make human cell sources preferred for drug permeability screening due to the presumed physiological relevance to the patient. Primary cells, directly isolated from patients, often present a phenotype most similar to in vivo, but are often difficult to acquire due to ethical reasons, require intricate isolation protocols, and present concerns with patient specific differences. Therefore, much of the emphasis has been placed on establishing and characterizing human immortalized cell lines for robust screening methods.

Since its establishment in 2005, the human cerebral microvessel endothelial cell line (hCMEC/D3) has been the most widely used immortalized endothelial cell line for BBB in vitro models (Weksler B et al., Fluids Barriers CNS 2013; 10:16). Although it is widely used, studies (as well as our observations, unpublished results) have revealed that hCMEC/D3 cells can have relatively “leaky” tight junctions and demonstrate a functional reduction in efflux transporter expression with passaging (Biemans Ealm, et aL, J Neurosci Res 2017; 95:1513-1522; Urich E et al., PloS One 2012; 7:e38149). The hCMEC/D3 cells were also isolated from a single patient who suffered from epilepsy and was immortalized by a co-transfection of hTERT oncogene and SV40. An alternative immortalized human brain endothelium is the HBEC-5i cell line that was singly transfected with SV40 and originates from a patient pool of cerebral cortex fragments, lacking pathological abnormalities. The HBEC-5i has been used predominantly in the study of cerebral malaria; however, these studies have established the potential for this cell line to be used for BBB in vitro permeability screening (Wassmer S C et al., Infect Immun 2006; 74:645-653). These cells have been observed to express a high number of electron-dense tight junctions as seen under electron scanning microscopy, as well as provide high transendothelial electrical resistance (TEER) and low permeability comparable to other immortalized BMECs. Recently, the HBEC-5i cell line has been used for in vitro modeling of the BBB showing functional expression of ABC transporters and stable barrier properties over multiple days of culture, suggesting that they are a viable alternative to the hCMEC/D3 cell line and other immortalized BMEC sources (Puech C et al., Int J Pharm 2018; 551:281-9; Puech C et al., Brain Res 2019; https://doi.org/10.1016/j.brainres.2019.05.024).

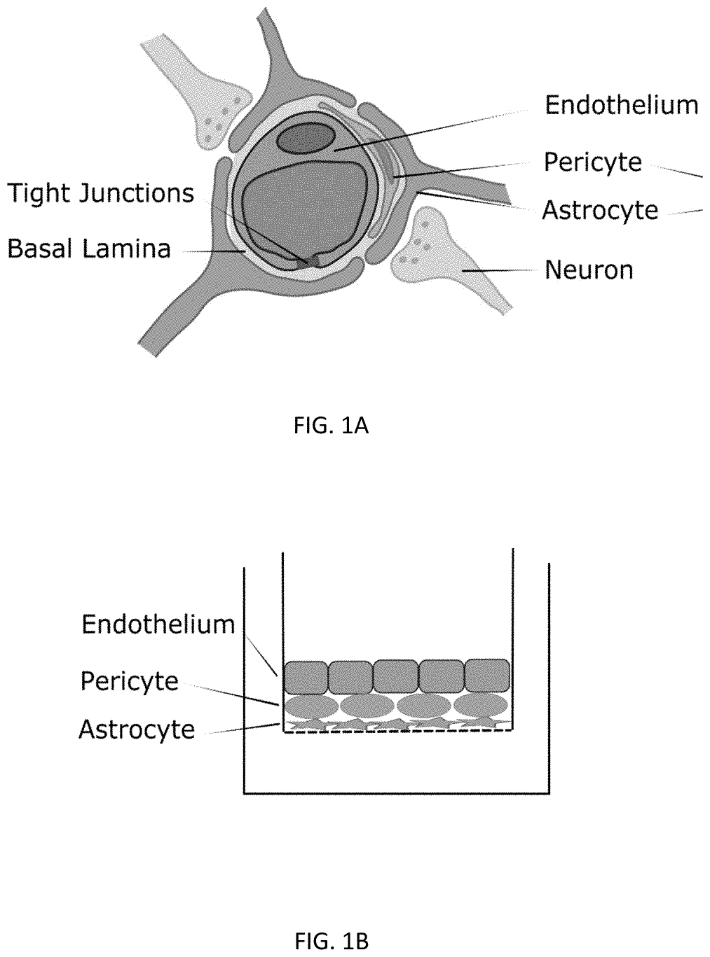

Given the interaction of multiple cell types in the NVU to maintain the BBB phenotype, many in vitro models include astrocytes and pericytes in conjunction with BMECs. Typically, these models involve seeding the endothelium on the apical surface of the filter and the supporting NVU cells in the basolateral chamber or on the reverse side of the filter. Seeding supporting NVU cells on the reverse side of the filter support displays improved barrier properties in the cultured BMECs by reducing the distance between the cell types and further enhances the BBB phenotype in the cultured endothelium (Gaston J D et al., J Healthc Eng 2017;2017:.https://doi.org/10.1155/2017/5740975). However, the direct cell-cell contact is limited due to the thickness of the filter support and opposable culturing surfaces, where growth through the filter pores provide limited interactions. Studies in our laboratory have demonstrated that seeding endothelium directly layered atop a lawn of cultured astrocytes results in direct cell-cell contacts that enhance barrier properties in comparison to indirect culturing methods (Kulczar C, et al., J Pharm Pharmaol 2017; 69:1684-1696). In this study we have further developed and optimized the direct contact, layered coculture model to a triculture system with the inclusion of pericytes to further increase the physiological relevance of the in vitro model. The direct contact, layered triculture model is cultured by seeding astrocytes, followed by pericytes, then the endothelium all on the apical side of a filter support to reflect the in vivo configuration and cell-cell contacts of the NVU (FIG. 1). In our previous studies, we have utilized a One Factor at a Time approach to optimize culturing variables in a laborious and time-consuming manner. Given the multiple factors that influence the performance of this model, we have now utilized a method of Design of Experiments (DOE) approach to determine optimal culturing conditions by assessing the influence of multiple variables on barrier properties in a single experiment. This study has demonstrated that a DOE based approach, typically utilized in non-biological process optimization, can be used to optimize other multi-factor cell-based in vitro systems by assessing variable influence on model performance. Additionally, the results of this study demonstrate the importance of direct cell contact in in vitro models and suggests that increasing physiological relevance of in vitro models to mimic the in vivo NVU can further enhance screening tools for neurotherapeutic development.

Materials and Methods

TRANSWELL® filters of 12 mm 0.4 μm pore size, T-75 culture flasks, MATRIGEL®, mouse laminin, and type I rat tail collagen were purchased from Corning (Corning, N.Y., USA). Hank's balanced salt solution (HBSS) and Dulbecco's Modified Eagle Medium/Nutrient Mixture F-12 (DMEM/F-12) were obtained from Gibco (Carlsbad, Calif., USA). Fetal bovine serum (FBS), hydrocortisone, lithium chloride, retinoic acid, rhodamine 123 (R123), elacridar, digoxin, carbamazepine, colchicine, clozapine, caffeine, and prazosin hydrochloride were purchased from MilliporeSigma (St. Louis, Mo., USA). HEPES (2-[4-(2-hydroxyethyl)piperazin-1-yl]ethanesulfonic acid) and calcium chloride dihydrate were obtained from J.T. Baker (Phillipsburg, N.J., USA). Dexamethasone was obtained from MP Biomedicals (Santa Ana, Calif., USA). Endothelial cell growth supplement (ECGS) was purchased from Alfa Aesar (Haverhill, Mass., USA). Fluorescein isothiocyanate (FITC) labeled 4 kD dextran was purchased from Chondrex (Redmond, Wash., USA). Poly-L-lysine (PLL) was purchased from Trevigen (Gaithersburg, Md., USA). Radiolabeled compounds [14C]-mannitol, -sucrose, -inulin, -PEG-4000, and [3H]-L-histidine were purchased from Moravek Biochemicals Inc. (Brea, Calif., USA). Human astrocytes, human brain vascular pericytes, astrocyte medium, pericyte medium, and astrocyte and pericyte growth factors were all obtained from ScienCell Research Laboratories (Carlsbad Calif., USA). HBEC-5i cells were purchased from ATCC (Manassas, Va., USA).

Cell Culture. Human Brain Endothelial Cells (HBEC-5i) were maintained in T-75 culture flasks pre-coated with Type I rat tail collagen with medium changes every 3 days and culturing at 80-90% confluency. The cells were utilized in the studies between passages 22 and 30. HBEC-5i culture medium was made up of Dulbecco's Modified Eagle Medium/Nutrient Mixture F-12 (DMEM/F-12) supplemented with 10% FBS, 15 mM HEPES, and 40 ug/mL endothelial cell growth supplement (ECGS). Human astrocytes and human brain vascular pericytes are maintained in T-75 culture flasks pre-coated with poly-L-lysine with medium changes every 3 days and subculturing at 80-90% confluency. For the studies presented herein, the astrocytes and pericytes were utilized between passage 4 and 10. Astrocyte culture medium was made up of Astrocyte Medium supplemented with 5% FBS, astrocyte growth supplement, and penicillin/streptomycin. Pericyte culture medium was made up of Pericyte Medium supplemented with 5% FBS, pericyte growth supplement, and penicillin/streptomycin.

Experimental Design for Optimization. Optimization of plating conditions (cell seeding densities, extracellular matrix protein, and length of culture) and medium additives were performed in sequential design of experiment (DOE) analyses. For plating studies JMP® 13.2 from SAS statistical software was used to determine the plating conditions of each experimental run for a total of 39 combinations by utilizing a 5 factor, 2 level, custom design (DOEₚ). Each run was done in a single replicate with DOEₚ selected conditions to determine best levels for each variable and then the combined optimized conditions were further confirmed in subsequent experiments in triplicate. Table 1 lists the various factors and the respective levels of each.

TABLE 1
Plating Factors and Conditions for DOE_{P}

| Factor | Selected Range* | |
|---|---|---|
| Astrocyte Seeding Density | 20,000-60,000 | cells/cm² |
| Pericyte Seeding Density | 20,000-60,000 | cells/cm² |
| HBEC-5i Seeding Density | 50,000-110,000 | cells/cm² |

| | |
|---|---|
| Study Day | Day 5-9 |
| Extracellular Matrix | Collagen I, Matrigel, Laminin |

*3 levels each factor

TABLE 2
Optimization with Evaluation on Day 5 and 7 (DOE_{M2})

| Factor+ | Selected Range* | |
|---|---|---|
| Hydrocortisone | 0-1.4 | μM |
| Dexamethasone | 0-10 | μM |
| Lithium Chloride | 0-10 | mM |
| Retinoic Acid | 0-10 | μM |

| | |
|---|---|
| Study Day | Day 5 or 7 |

*2 levels each factor (presence of absence of given additive)

TABLE 3
Medium Optimization with Evaluation on Day 9 (DOE_{M1})

| Factor | Selected Range* | |
|---|---|---|
| HEPES | 15-25 | mM |
| Hydrocortisone | 0-1.4 | μM |
| Dexamethasone | 0-10 | μM |
| Lithium Chloride | 0-10 | mM |
| Calcium | 0-1 | mM |
| Retinoic Acid | 0-10 | μM |

| | |
|---|---|
| Study Day | Day 9 |

*2 levels each factor (presence of absence of given additive)
+ All medium supplemented with 15 mM HEPES

Similarly, medium optimization was performed in two analyses using a custom design DOE to determine medium conditions that resulted in the tightest barrier properties. The first analysis (DOE_{M1}) was performed using HEPES, hydrocortisone, dexamethasone, LiCl, calcium, and retinoic acid—observing permeability at 9 days post endothelial cell plating (Table 2). A second analysis (DOE_{M2}) was performed, based on the results of the first, using hydrocortisone, dexamethasone, LiCl, and retinoic acid at both 5 and 7 days post endothelial cell plating (Table 3).

Plating Direct Contact Triculture on TRANSWELL® Filter Support

For the DOEₚ studies, filters were pre-coated with poly-L-lysine (PLL) by pre-coating 12 mm, 0.4 μm pore TRANSWELL® inserts with 5 μg/cm² PLL. Astrocytes were plated at seeding densities of 20,000, 40,000, or 60,000 cells/cm² and allowed to grow for 48 hours. After 48 hours of astrocyte growth, astrocyte medium was removed and pericytes were seeded atop the astrocyte lawn at seeding densities of 20,000, 40,000, or 60,000 cells/cm² and allowed to grow for 48 hours. After 48 hours of pericyte growth, apical medium was replaced with the specified ECM protein solution. Astrocyte-pericyte lawn filters were coated with one of the following ECM proteins at the respective concentrations: MATRIGEL®, 25 μL/cm² (2.5 μg/cm²), Laminin 5 μg/cm², or Type I Rat Tail Collagen 5 μg/cm². To coat inserts, MATRIGEL®, Laminin, or collagen I aliquots were diluted in HBSS with Ca²⁺ and Mg²⁺ and 0.5 mL dispensed onto to each respective 12 mm insert. Inserts were left to incubate with the respective ECM protein for 45 min at 37° C. After incubation, the ECM solution was removed and HBEC-5i cells were plated at seeding densities of 50,000, 80,000, or 110,000 cells/cm² and allowed to grow for 5, 7, or 9 days prior to permeability measurements. Cultures were maintained in complete HBEC-5i medium with medium changes every other day following endothelial cell plating. Transendothelial electrical resistance (TEER) was measured every 24 hours after HBEC-5i plating using 4 mm Chopstick electrode with EVOM2 Volt/Ohm Meter (World Preclinical Instruments), and normalized based on resistance across blank filter supports.

In DOE_{M1/2} studies, culturing methodology described above was used with the modification that the complete HBEC-5i culture medium was supplemented with additional factors and introduced to cultures 24 hours post endothelial plating with medium changes every other day until the day of study. Medium for DOE_{M1/2} was prepared from concentrated stock solutions of 1 M HEPES in water, 4.6 mM hydrocortisone in ethanol, 3.8 mM dexamethasone in DMSO, 11.8 M LiCl in water, 1.7 M CaCl₂ in water, and 33.3 mM retinoic acid in DMSO. Total percentage of each solvent was kept constant across all medium conditions.

Plating Monoculture and Direct Contact Coculture on TRANSWELL® Filter Support

Monoculture (HBEC-5i alone) and direct contact coculture (astrocyte-HBEC-5i and pericyte-HBEC-5i) models were used for comparison with the direct contact triculture. For monoculture studies, 12 mm, 0.4 μm pore TRANSWELL® inserts were pre-coated with 25 μ/cm² MATRIGEL®. HBEC-5i cells were plated on MATRIGEL® coated filters at a density of 80,000 cells/cm² and cultured for 9 days with medium changed every other day. Direct contact cocultures were plated according to methods developed by Kulczar et al. with some modifications.⁵⁵ TRANSWELL® filters were pre-coated with 5 μg/cm² PLL followed by seeding of astrocytes or pericytes at 20,000 cells/cm² and allowed to grow for 48 hours. At 48 hours post astrocyte or pericyte plating, HBEC-5i cells at 80,000 cells/cm² were seeded directly atop the lawn of pre-seeded cells and cultured for an additional 9 days with medium changed every other day.

Permeability Assays

To optimize conditions permeability was measured using 4 kD FITC-dextran at an initial concentration of 0.25 mg/mL in HBSS with Ca²⁺ and Mg²⁺. Tricultures were washed and left to equilibrate in HBSS at 37° C. for 30 minutes prior to the start of the permeability assay. Permeability was performed at 37° C. on a rocking platform maintaining sink conditions and sampling at 15, 30, 45, 60, and 90 minutes. Samples of 100 μL from each basolateral chamber were removed at each time point and placed into a 96-well black flat-bottomed well plate for fluorescence reading. Samples were analyzed using a BioTek Synergy 4 plate reader at excitation of 485 nm and emission of 530 nm. Apparent permeability (Pₐₚₚ) was calculated using the following equation 1 (eq. 1)

$${P}_{app} = \frac{dM / dT}{{C}_{0} \times A} \qquad ( eq . 1 )$$

wherein dM/dT is the amount of Dextran that moves across the filter over time, Co is the initial concentration in the donor (apical) chamber, and A is the surface area of the filter support. The effective permeability (P_{eff}, permeability contributions of cell layer alone) of each condition was determined using the following equation 2 (eq. 2)

$$\frac{1}{{P}_{app}} = \frac{1}{{P}_{eff}} + \frac{1}{{P}_{filter}} \qquad ( eq . 2 )$$

wherein the P_{filter} value used is that of the ECM used in the given condition.

Apparent permeability of additional paracellular markers of varying sizes ([¹⁴C]-mannitol, [¹⁴C]-sucrose, [¹⁴C]-inulin, and [¹⁴C]-PEG-4000) was determined in the optimized direct contact triculture. Permeability assays were performed as stated above with an initial concentration of 0.25 μCi/mL in HBSS for all markers and analysis performed by liquid scintillation counting.

A range of BBB positive and negative permeants were used to further evaluate barrier properties of the optimized model. The permeability of [³H]-L-histidine, carbamazepine, colchicine, digoxin, clozapine, and prazosin was determined by preparing 10 mM stock solutions of each compound in DMSO, with the exception of [³H]-L-histidine. For each study, the final concentration of DMSO was equivalent at 1% (v/v). Permeability of [³H]-L-histidine was determined using the same method as stated above for radiolabeled paracellular markers. Working solutions of non-radiolabeled compounds were prepared at a concentration of 25 μM in HBSS with permeability measurements performed as stated above and sampling at 30, 60, 90, 120, and 150 minutes. Analysis for these compounds was performed using high performance liquid chromatography (HPLC). Permeability was calculated according to equation 1.

The function of P-gp in the triculture model was determined using P-gp substrate rhodamine 123 (R123) in the presence and absence of the inhibitor elacridar. Stock solutions of R123 (2 mM) and elacridar (10 mM) were prepared in DMSO. Working solutions of 10 μM R123 and 2 μM elacridar were prepared in HBSS with 1% DMSO. For inhibition studies, tricultures plated on permeable filter supports were pre-incubated with 2 μM elacridar for 45 minutes prior to the addition of R123. Samples were removed at 30, 60, 90, and 120 minute time points and analysis was performed using the BioTek Synergy 4 plate reader at excitation of 485 nm and emission of 530 nm. Permeability was calculated according to equation 1.

High Performance Liquid Chromatography

Analysis of carbamazepine, caffeine, colchicine, digoxin, clozapine, and prazosin was performed on an Agilent 1100 reverse phase HPLC with variable wavelength detection (VWD). All samples were run isochratically through an ASCENTIS® C-18 15×4.6 mm, 5 μm column, at 25 μL injection volume, using water and acetonitrile (ACN) for all mobile phase. Carbamazepine analysis was performed using a column temperature of 40° C., mobile phase of 65:35, water:ACN, at a 1.5 mL/min flow rate, and absorbance measurement at 284 nm. Caffeine analysis was at ambient temperature, a mobile phase of 90:10, water:ACN, at a 1.0 mL/min flow rate, and absorbance measurement at 275 nm. Colchicine analysis was performed using a column temperature of 40° C., mobile phase of 75:25, water:ACN, at a 1.5 mL/min flow rate, and absorbance measurement at 354 nm. Digoxin was analyzed using a column temperature of 40° C., a mobile phase of 70:30, water:ACN, at a 1.1 mL/min flow rate, and absorbance measurement at 218 nm. Clozapine analysis was performed using a column temperature of 40° C., mobile phase of 45:55, water:ACN, at a 1.5 mL/min flow rate, and absorbance measurement at 254 nm. Prazosin analysis was performed using a column temperature of 40° C., mobile phase of 65:35, water:ACN, at a 1.5 mL/min flow rate, and absorbance measurement at 254 nm.

Statistical Analysis

JMP 13.2 statistical software was used to generate custom experimental designs based on categorical and discrete continuous factors. Analysis of each DOE was done by fitting models based on the P_{eff} of 4 kD dextran response to standard least squares to determine optimal conditions. In comparison studies, all conditions were performed in triplicate (n=3) and subjected to Student's t-test or one-way ANOVA with Tukey Kramer post-hoc test. A p-value of 0.05 was considered to be statistically significant.

Results

Plating Optimization (DOEₚ)

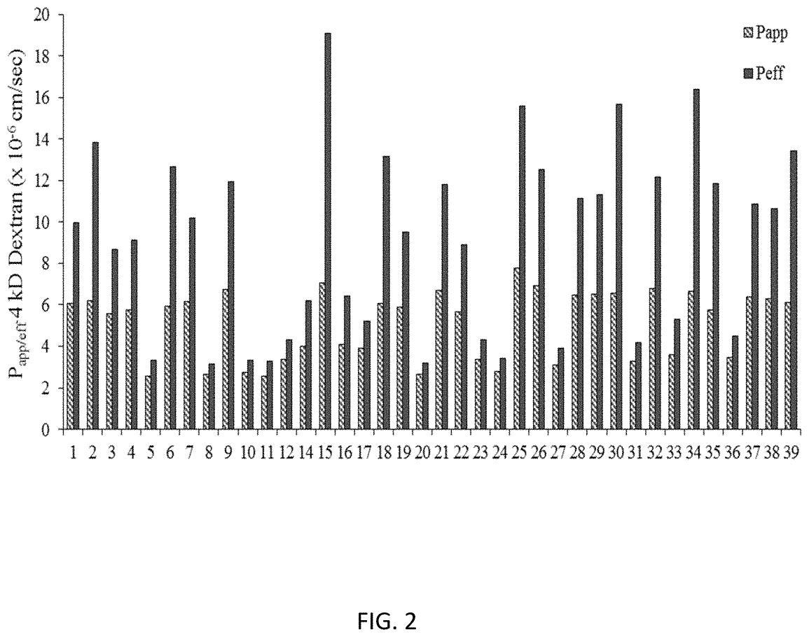

Traditionally, a one factor at a time (OFAT) approach is used for assessing the impact of variable changes in biologically based models and processes, where one variable (e.g. cell density) is optimized in the presence of several other unoptimized variables in an inefficient and laborious manner. A design of experiments based approach allows for the influence of multiple factors to be observed on a measured response to arrive at an optimal level for each given variable. Furthermore, it allows one to more rapidly identify optimized growth conditions in a time and labor efficient manner. Based on previous studies establishing a direct contact triculture and our direct contact coculture model, informed selection of the seeding densities of all three cell types, ECM used to aid endothelial attachment, and length of culture of the endothelium were the selected factors. Optimal plating conditions were determined using P_{eff} values to account for the differences associated with ECM coatings. Conditions 8 (60 HA, 60 HBVP, 110 EC, Laminin, Day 9) and 20 (20 HA, 20 HBVP, 110 EC, Laminin, Day 9) exhibited the lowest P_{eff} values at 3.2×10⁻⁶ cm/sec (FIG. 2).

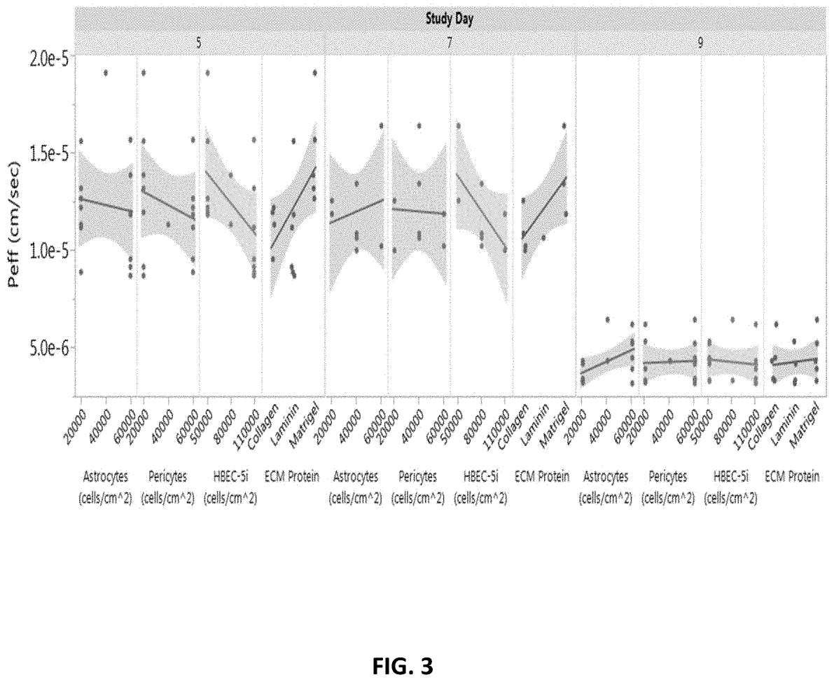

Based on the trends in the data, study day has the largest impact on paracellular permeability resulting in significantly lower 4 kD dextran permeability at day 9 compared to days 5 and 7. When separating the data by study day and factor there are observable trends in the permeability, including the effects of astrocyte and pericyte cell density. With extended culturing, higher seeding densities of astrocytes appears to result in higher permeability of the dextran, however this observation is not significant (FIG. 3). HBEC-5i seeding density also shows trends towards lower permeability at higher seeding densities; however, this trend is not as strong at day 9 when the cells have had sufficient time to reach confluency.

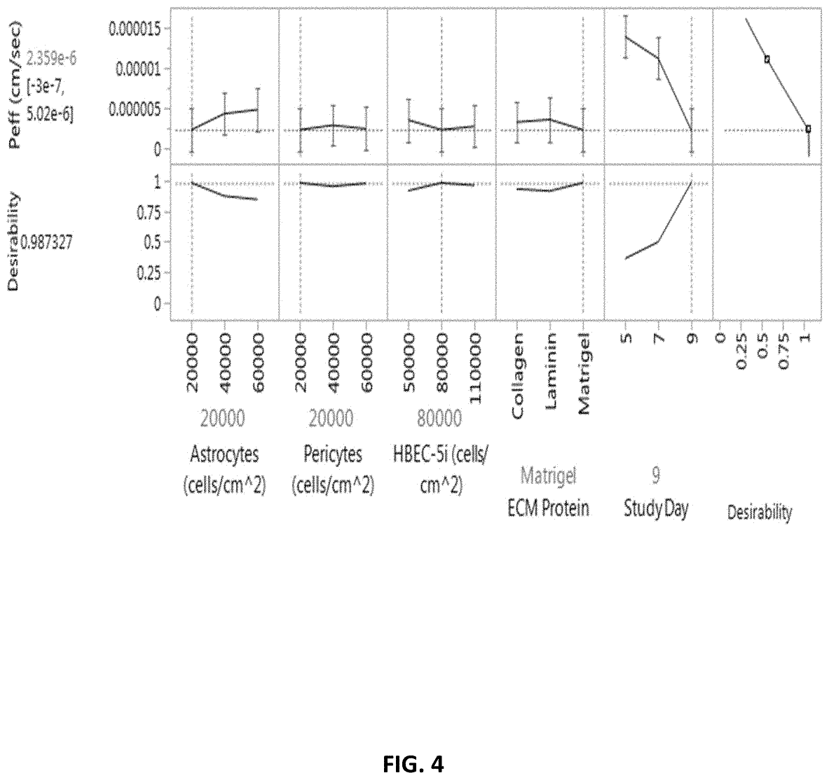

Using JMP 13.2 software, a prediction profiler was generated based on the obtained P_{eff} values for the given conditions. By maximizing the Desirability to achieve the lowest possible permeability, the optimal conditions were determined to be 20,000 cells/cm² for both astrocytes and pericytes, 80,000 cells/cm² HBEC-5i cells, MATRIGEL® as the ECM protein, and culturing for 9 days post endothelial cell plating (FIG. 4). These conditions would optimally generate a predicted P_{eff} value of 2.4×10⁻⁶ cm/sec for 4 kD dextran. Upon repeating the analysis at selected optimal conditions, the P_{eff} of a 4 kD dextran showed to be reproducible resulting in a similar permeability value (P_{eff}; 3.7×10⁻⁶ cm/sec ±0.04, n =3).

Medium Optimization (DOE_{M1/2})

Selection of medium additives were chosen based on literature and previous studies in our laboratory to be added to HBEC-5i medium based on their reported influences on barrier tightness both in vitro and in vivo (Mizee M R et al., J Neurosci 2013; 33:1660-1671). Unmodified HBEC-5i medium contains 15 mM HEPES; therefore, higher levels of HEPES were included to assess the impact that a higher buffering capacity would have on barrier tightness. Hydrocortisone was selected for its influence on inflammatory responses as a glucocorticoid and potential to prevent tight junction break down. Lithium chloride has been shown to influence claudin expression through stimulation of the Wnt/≢-catenin pathway.⁵⁷ Calcium was studied as a medium additive due to its influence on adherens and tight junction protein expression to increase barrier tightness, where studies have shown that low extracellular calcium levels can lead to an increase in paracellular permeability. Like hydrocortisone, dexamethasone acts to inhibit inflammatory responses and upregulate tight junctions; however, it is a synthetic alternative to the naturally occurring hydrocortisone. Lastly, retinoic acid is naturally secreted by glial cells and has revealed significant increases in paracellular tightness in in vitro BBB models (Lippmann ES et al., Sci Rep 2014; 4:4160).

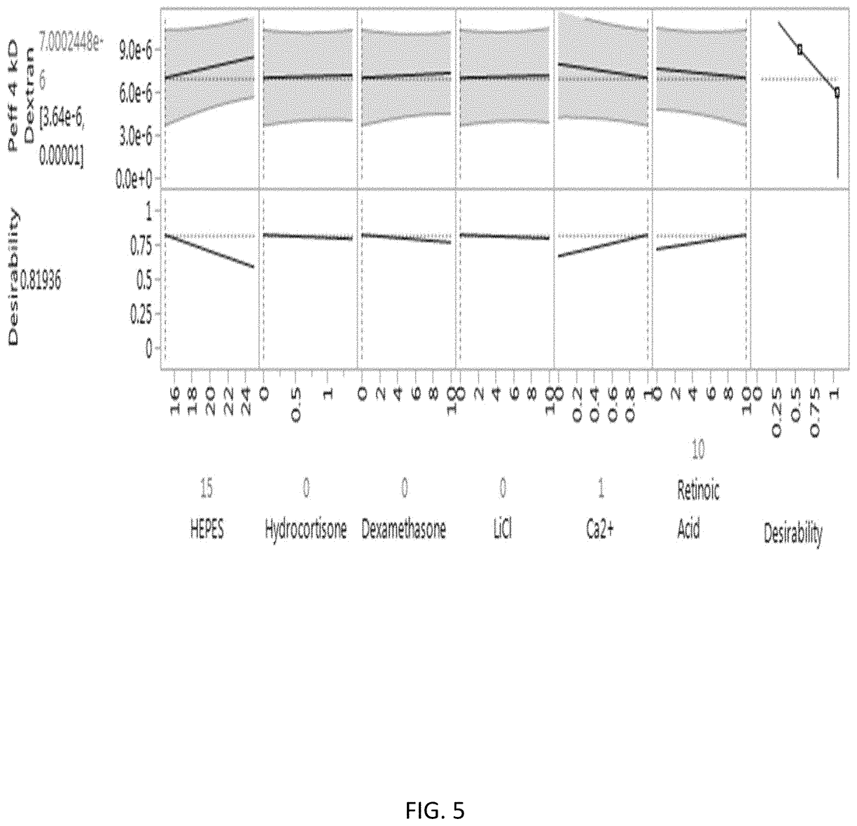

The first analysis of medium optimization (DOE_{M1}) was performed at optimal plating conditions determined from DOEₚ: 20,000 cells/cm² for astrocytes and pericytes, 80,000 cells/cm² HBEC-5i, MATRIGEL®, after 9 days of endothelial growth. HEPES, hydrocortisone, dexamethasone, lithium chloride, calcium, and retinoic acid were chosen as medium additives due their reported influence on tight junction expression and induction of barrier properties in in vitro BBB models. The lowest achieved 4 kD dextran P_{eff} of DOE_{M1} was 6.3×10⁻⁶ cm/sec, suggesting that, under these conditions, the additives did not provide further tightening of the model. Strong trends are not apparent for any of the additives with the exception of higher levels of HEPES resulting in higher permeability values. The optimal medium condition was determined to be 15 mM HEPES, 1 mM calcium, and 10 μM retinoic acid, but the influence of these factors on barrier tightness was not significant (FIG. 5).

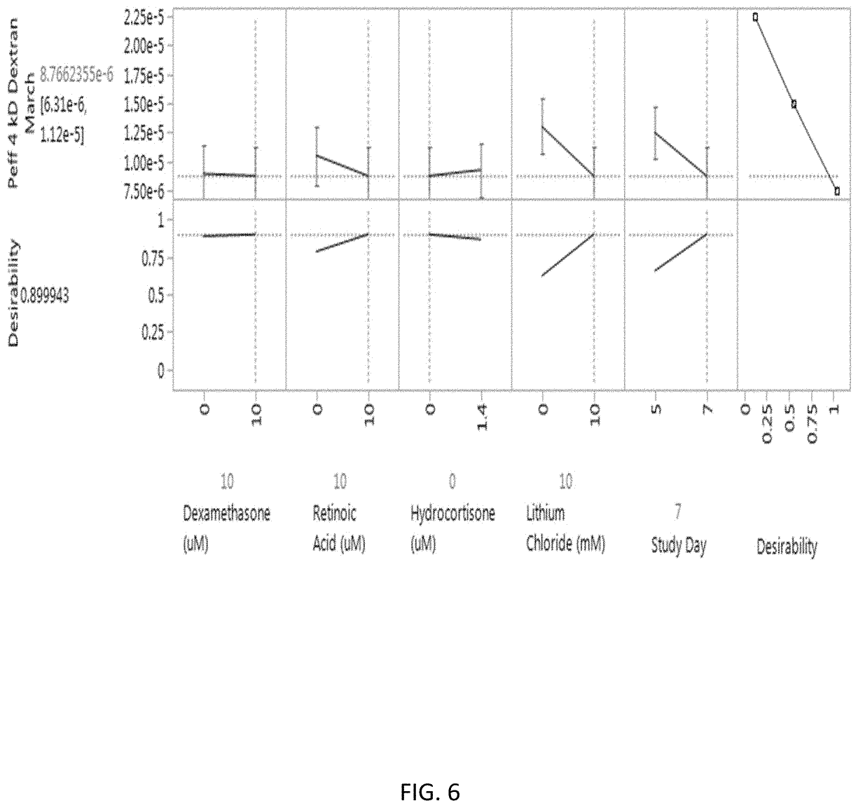

Based on these results a second analysis (DOE_{M2}) was performed to assess the influence of the additives in earlier days of culture. These studies were conducted in the presence or absence of hydrocortisone, dexamethasone, lithium chloride, and retinoic acid at 5 and 7 days post endothelial cell culture, HEPES was held constant at 15 mM and calcium was removed from DOE_{M2}. The lowest 4 kD dextran P_{eff} of DOE_{M2} was 8.3×10⁻⁶ cm/sec, suggesting that the additives do not provide increased barrier tightness based on the optimized plating conditions of DOEₚ. Optimal conditions for medium was determined to be 10 μM dexamethasone, 10 μM retinoic acid, 10 mM LiCl, through 7 days of endothelial cell culture; however, these conditions were not used for continued assessment of the optimized model due to the lack of improvement over unmodified medium (FIG. 6).

Comparison to Mono- and Cocultures

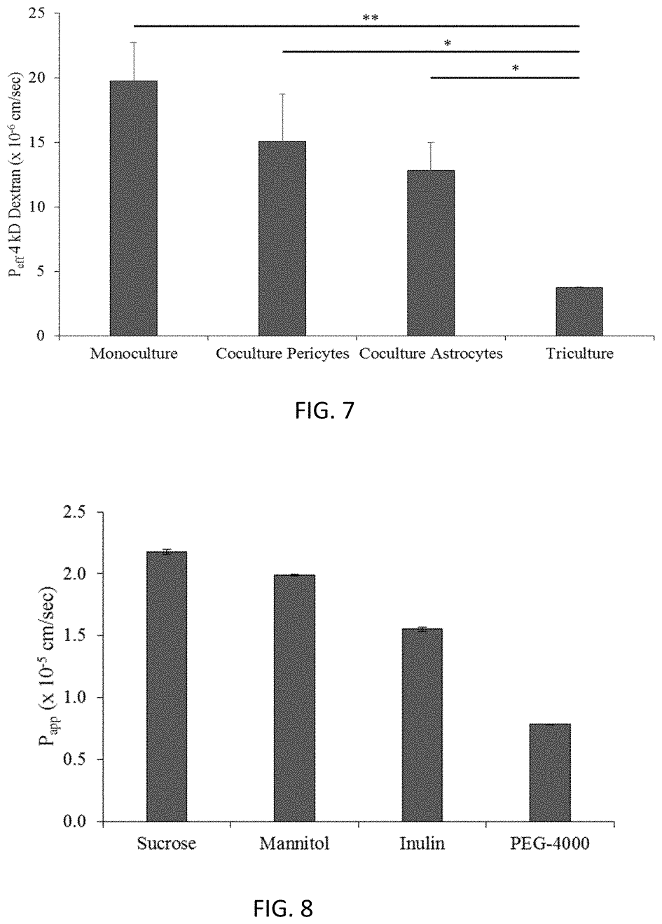

The optimized direct contact triculture was compared to a monoculture of HBEC-5i cells alone and direct contact cocultures of HEBC-5i cells plated atop a lawn of astrocytes or pericytes (FIG. 7). Effective permeability of the 4 kD FITC-dextran was used for comparison between the different models. In comparison to the optimized direct contact triculture (3.7×10⁻⁶±0.0 cm/sec) the HBEC-5i monoculture had the highest observed permeability (19×10⁻⁶±3.0 cm/sec; p<0.01), followed by the perictye-HBEC-5i coculture (15.1×10⁻⁶±3.7 cm/sec; p<0.05), and the astrocyte-HBEC-5i coculture (12.8×10⁻⁶±2.1 cm/sec; p<0.05). Given the significant differences observed between the direct contact triculture and the monoculture and coculture models, the inclusion of all three cell types offers increased barrier tightness for the in vitro model.

Direct Contact Triculture BBB Marker Compounds

Paracellular markers possessing a broad range of hydrodynamic radii were used to evaluate the functional tightness of the optimized model (FIG. 8) (Ghandelari H et al., J Pharmacol Exp Ther 1997; 280:747-53). The lowest apparent paracellular permeability observed was that of PEG-4000 (0.78×10⁻⁵±0.00 cm/sec, 15.9 Å) followed by inulin (Pₐₚₚ=1.55×10⁻⁵±0.01 cm/sec, 10 Å), mannitol (Pₐₚₚ=1.99×10⁻⁵±0.01 cm/sec, 4.3 Å), and sucrose (Pₐₚₚ=2.18×10⁻⁵±0.02 cm/sec, 5.2 Å). The apparent paracellular permeability of the hydrophilic markers shows the model is able to distinguish between markers of varying sizes. However, based on the hydrodynamic radius, sucrose should have a lower permeability as the larger compound in comparison to mannitol.

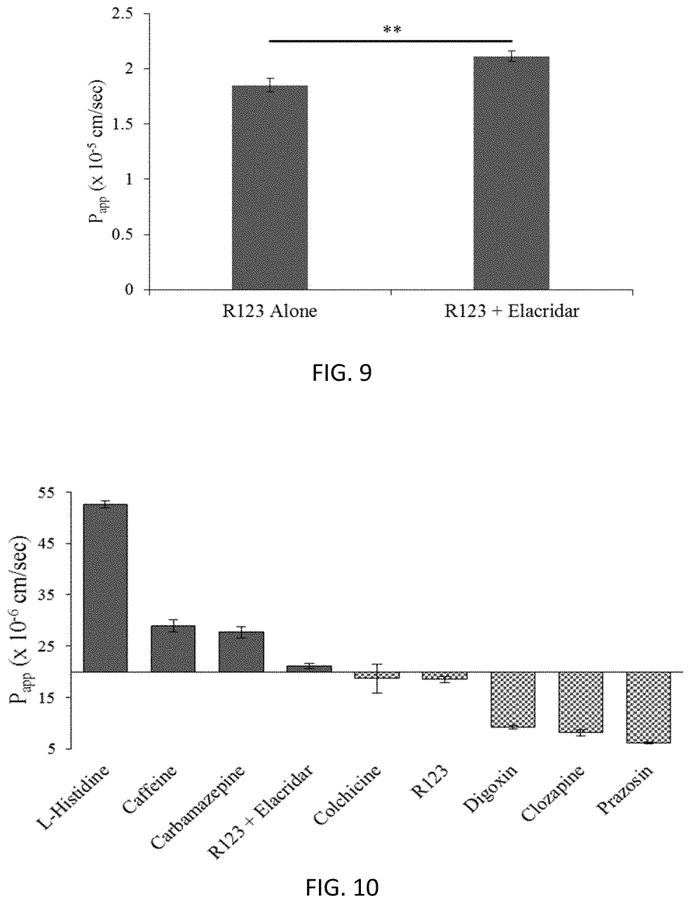

P-gp function in the direct contact triculture was assessed using P-gp substrate R123 alone and in the presence of P-gp inhibitor elacridar (FIG. 9). In the absence of inhibitor, the Pₐₚₚ of R123 was 18.52×10⁻⁵±0.58 cm/sec. The presence of elacridar significantly increased the Pₐₚₚ of R123 (Pₐₚₚ=21.14×10⁻⁶±0.46 cm/sec; p<0.01) across the direct contact triculture. Additional P-gp substrates were utilized as marker compounds such as digoxin (Pₐₚₚ=9.21×10⁻⁶±0.31 cm/sec) and colchicine (Pₐₚₚ=18.67×10⁻⁶±2.75 cm/sec). Prazosin, a BCRP substrate, was used to assess the function of other efflux transporters in the direct contact model (Pₐₚₚ=6.16×10⁻⁶±0.11 cm/sec) (FIG. 10).

The antipsychotic drug clozapine showed an apparent permeability value of 8.15×10⁻⁶±0.58 cm/sec. The amino acid L-histidine was used to assess facilitative transport across the in vitro model with an observed apparent permeability of 52.61×10⁻⁶±0.70 cm/sec, as reported previously (Carl SM et al., Mol Pharm 2010; 7:1057-1068). Carbamazepine is an antiepileptic drug and a BBB positive permeant with an observed apparent permeability of 27.71×10⁻⁶±1.13 cm/sec in the optimized model. Caffeine, a small hydrophilic molecule, also had BBB positive permeation with an obtained apparent permeability of 28.93×10⁻⁶±1.15 cm/sec (FIG. 10).

Discussion

The HBEC-5i cell line has not been as extensively used for in vitro BBB permeability modeling comparative to other BMEC cell sources (e.g. hCMEC/D3). However, it has been shown to have good expression levels of brain endothelial markers such as vascular cell adhesion molecule (VCAM-1) and intercellular adhesion molecule (ICAM-1) essential for immune cell trafficking, CD51 (αᵥ-integrin) that is involved in extracellular matrix adhesion, as well as tight junction proteins zonula occluden 1 (ZO-1) and claudin-5 (Wassmer S C et al., Infect Immun 2006; 74:645-653). Transporter expression and function of BCRP, P-gp, MRP-1, and MRP-2 has also been recently evaluated and shown to be comparable to other immortalized brain endothelium. Conversely, this cell line has also been indicated to be lacking in expression of platelet endothelial cell adhesion molecule (PECAM-1, CD31) and CD36. Given the expression of endothelial markers and transporters that have been investigated by others, we selected the HBEC-5i cell line as the BMEC for the direct contact triculture over using the hCMEC/D3 cell line that we utilized in the development of the direct contact coculture (Kulczar C et al., J Pharm Pharmacol 2017; 69:1684-96).

In vitro models of the BBB are increasingly being developed to provide physiological relevance through co- and triculture indirect contact methods with astrocytes and pericytes that comprise the NVU to further enhance barrier properties. However, the direct cell-cell contacts of astrocytes and pericytes with the endothelium in vivo are often overlooked in these multi-cellular models that are currently being utilized. We have previously shown that the direct contact between astrocytes and the endothelium in a coculture model increases the barrier properties compared to endothelial monocultures and indirect plating methods. Although astrocytes are often used in in vitro models as a supporting cell, pericytes also play an important role in influencing and regulating the BBB phenotype through a number of signaling cascades. Since each supporting cell acts in a functionally different manner on the BMECs, incorporating both astrocytes and pericytes in direct contact cell based models should better enable synergistic effects of the NVU to be represented in vitro.

A design of experiments approach was taken to develop and optimize the direct contact triculture in order to adequately understand the interactions each variable would have on the performance of the model. As opposed to an OFAT approach, DOE takes into account the implications of changing multiple variables to come to optimal conditions in a significantly more efficient manner in terms of time invested and resources required. In optimizing the triculture we arrived at optimal conditions with reproducible results in a time frame of two months as opposed to our previous optimization efforts that spanned the course of multiple years. The results of DOEₚ revealed optimal plating conditions of 20,000 cells/cm² for both astrocytes and pericytes, 80,000 cells/cm² for HBEC-5i, MATRIGEL® as the ECM to promote endothelial adhesion, and culturing the endothelium for 9 days after seeding. The comparison of 4 kD dextran permeability to other reported data revealed that our optimized model infers that the model is among the tightest we found reported, suggesting that culturing multiple NVU cell types in direct contact synergistically increases barrier tightness (Table 4).

In addition to selecting an optimized set of plating conditions, the DOE approach facilitated an understanding of how changing factor levels may impact the model performance. At higher densities of astrocytes and pericytes a decrease in paracellular tightness was observed with extended culture time. This phenomenon is likely due to the length of time these cells are in culture, with the astrocytes and pericytes possibly becoming senescent by the day of study. Additionally, higher seeding densities of endothelial cells resulted in lower paracellular permeation rates at day 5 and 7, which may be expected by the increased ability of the cells to form a confluent layer at fewer days of culture. However, that trend is less drastic after 9 days of culture suggesting that seeding density does not play as significant of a role at confluency, but rather time in culture is necessary to allow for differentiation and adequate tight junction formation.

TABLE 4
P_{eff} Values of 4 kD Dextran for Different BBB Models

| Model/Endothelial Cell Line | Peff (10⁻⁶ cm/sec) |
|---|---|
| DOE Direct Contact Triculture, HBEC-5i | 3.7 |
| Monoculture (HA conditioned medium), HBEC-5i | 3.6^{a} |
| Monoculture, hCMEC/D3 | 8.8^{b}, 5.4^{c} |
| Isolated endothelial cells, rat | 1.0^{d} |
| In vivo microvessels, rat | 0.92^{e} |

^{a}Puech, C., et al., Int J Pharm (2018) 551(1) 281-289;
^{b}Forster, C., et al., J Physiol (2008) 589(7) 1937-1949;
^{c}Weksler, B., et al., FASEB J. (2005) 19(13) 1872-1874;
^{d}Watson, P.M.D., et al., BMC Neuroscience (2013) 14:59;
^{e}Yuan, W. et al., Microvasc Res (2009) 77(2) 166-173

An effort to optimize culture medium (DOE_{M1} and DOE_{M2}) was made to further increase barrier properties of the model through the inclusion of additives that have been shown to enhance the BBB phenotype in in vitro and in vivo studies. Between both assessments it was revealed that the length of culture time for the endothelium still had the largest impact on model performance regardless of additives (FIG. 6). Based on this finding it is possible that due to the influence the additives have on the endothelium the HBEC-5i cells are differentiating before reaching confluency, and this is not sustainable through the length of culture. This phenomenon could also explain why the effects of additives appear to be more extreme in DOE_{M2}, culturing for 5 or 7 days post endothelial plating, as the differentiation effects may be occurring earlier and not maintained through culture times for DOE_{M1}. A way to improve on this would be to include HBEC-5i seeding density as a factor in further assessments of medium additives. With the trends of DOEₚ establishing the positive impacts higher seeding densities have on model tightness, seeding at a higher density (greater than the optimized 80,000 cells/cm²) with differentiation inducing medium supplements may result in the tightest barrier formed and additionally reduce culturing time. An alternative would be to continue with optimized conditions of DOEₚ and include time of addition as a factor in further studies by introducing additives after the HBEC-5i have been in culture for more than 24 hours.

The influence of the medium additives may also extend beyond paracellular tightness. Hydrocortisone has been shown to increase barrier tightness through the upregulation of tight junction proteins, but has also been demonstrated to induce efflux transporter expression (Maines L W, et al., Neuropharmacology 2005; 49:610-7; Wedel-Parlow M V, et al., J Neurochem 2009; 111:111-8). Expression and function of ABC efflux transporters, specifically BCRP and P-gp, was also demonstrated to be influenced by the release of tumor necrosis factor-α (TNF-α) and subsequent inflammatory responses. However, hydrocortisone is a glucocorticoid that has been demonstrated to impact P-gp and BCRP expression by inducing anti-inflammatory responses. Therefore, in addition to the impact on paracellular tightness, the induction of efflux transporter expression should also be assessed by evaluating the time of addition of hydrocortisone to the culture medium.

The increase in physiological relevance of adding additional cell types of the NVU in direct contact with BBB endothelium provides increased barrier restrictive properties in comparison to the endothelium alone. Additionally, including both supporting cell types (astrocytes and pericytes) in direct contact with HBEC-5i cells results in increased barrier tightness compared to direct contact cocultures (astrocyte- and pericyte-HBEC 5i combinations alone). This finding suggests that including both the astrocytes and pericytes in in vitro models further synergistically enhances the properties of the BBB in addition to better representing the in vivo NVU. The inductive effects of astrocytes and pericytes and their roles in BBB maintenance have been well established; however, many of the models used for in vitro BBB permeability screening do not consider the direct contact the different cell types have with one another in vivo. By seeding astrocytes, pericytes, and the endothelium directly atop one another this model better mimics the 20 nm distance between the cell types due to the presence of the basal lamina that is seen in vivo (Mathiisen T M, et al., Glia 2010; 58:1094-103). Although indirect plating methods with cell types cultured on opposite sides of a 10 μm thick filter support also provide increased barrier properties over endothelial monocultures, the direct contact triculture is more physiologically relevant to the in vivo NVU and does not require manipulation of the TRANSWELL® system and potentially is more amenable to automation for higher capacity throughput screening assays.

Paracellular permeants of increasing hydrodynamic radius were selected to evaluate the tight junction formation in the direct contact model. With increasing marker size there is a related decrease in paracellular permeability due to the size of the molecule in relation to the pore size of the tight junctions formed between adjacent endothelial cells. Permeability of [¹⁴C]-PEG-4000 (15.9 Å) is the lowest of all markers used as expected followed by [¹⁴C]-inulin (10 Å). In studies with the optimized direct contact triculture model the permeability of [¹⁴C]-sucrose (5.2 Å) is faster than that of the smaller [¹⁴C]-mannitol (4.3 Å), which is opposite of what would be expected based on molecule size alone. One possible explanation is that the relative size of the two markers is small in comparison to the pore size of the model that elucidating differences in their respective permeation rates would not be observable. Alternatively, sucrose, a disaccharide of a fructose and glucose molecule linked via glycosidic bond, may serve as a substrate for active or facilitative nutrient transporters. For example, glucose permeation across the BBB has been reported to be modulated by several nutrient transporters, in particular the facilitative Glucose Transporter 1 (GLUT1) that is highly expressed in both BMECs and astrocytes. Several neurotherapeutics utilize a pro-drug approach where the agent is conjugated to glucose in an effort to enhance brain parenchymal exposure via GLUT1 (Patching S G et al., Mol Neurobiol 2017; 54:1046-77; Morgello S, et al., Glia 1995; 14:43-54).

Based on the structure of sucrose, the idea that there is some degree of nutrient transporter activity of the purported paracellular marker via the GLUT1 transporter is feasible. Therefore, we posit that the observed permeation rate for sucrose would be higher due to a potential transporter contribution that is not available for [¹⁴C]-mannitol in the optimized direct contact triculture. This theory is further exacerbated by the presence of astrocytes and pericytes on the apical side of the TRANSWELL® in the direct contact triculture since both of these cell types have reported expression of GLUT1, which may further increase the permeation of [¹⁴C]-sucrose in the apical to basolateral direction in comparison to in vitro models that culture these cells on the underside of the filter or in the basolateral chamber. The exact cause of the higher [¹⁴C]-sucrose permeability can be further investigated using GLUT1 or related transporter inhibitors or transfected HBEC-5i cells with modified expression of glucose transporters.

The functional activity of efflux transporters in BMECs is a key characteristic of the BBB, with the most prevalent isoform being P-gp. P-gp and related multidrug resistance conferring efflux transporters function to prevent xenobiotics from permeating into the brain parenchyma with a broad substrate affinity and capacity. Rhodamine-123 (R123) is a commonly used P-gp substrate to assess functional activity in the presence or absence of an inhibitor. Elacridar is a third generation P-gp inhibitor and has been reported to have among the highest specificity and potency for P-gp inhibition within the class of agents. We observed that the presence of elacridar resulted in an increase in R123 permeability across the direct contact triculture, suggesting that P-gp is functionally present in the optimized model. In these studies R123 permeation was only assessed in the apical to basolateral direction. Additional studies to elucidate P-gp function can include bi-directional permeability assessment as well as cellular accumulation; however, given the multiple cell types in direct contact the assessment of P-gp function an expression would require more in depth studies. This is particularly true given the fact that astrocytes have also been reported to express P-gp, which may further obfuscate P-gp assessment of the endothelium alone.⁷⁵

In addition to limiting paracellular permeation of hydrophilic solutes and potentially P-gp substrates, we theorized that a well-established in vitro model of the NVU should have an enhanced ability to differentiate between in vivo demonstrated high and low brain permeating compounds. In vitro permeability screening models capable of predicting in vivo permeation rates in order to rank new chemical entities is essential to facilitate compound advancement with translation as the aim. A number of positive and negative permeants were selected to assess the utility of the direct contact triculture. Amino acids and related analogues (e.g. γ-aminobutyric acid or GABA) play a critical role in maintaining brain homeostasis and modulating function. Here we selected L-histidine as an amino acid that is actively transported in a stereospecific manner across the BBB by amino acid transporters and potentially Peptide Histidine Transporter 1. However, L-histidine is a small water soluble molecule that can potentially permeate in vitro models to a significant extent via the paracellular pathway. Hence, the paracellular route may contribute to a higher permeation rate of L-histidine in comparison to other transporter specific markers. Caffeine was also selected as a small hydrophilic psychostimulant that has been demonstrated to permeate the in vivo BBB, and we demonstrated its permeation across the direct contact triculture model (Chen X et al., J Alzheimers Dis JAD 2010; 20:S127-41). Carbamazepine was selected as it is an anticonvulsant commonly used as a BBB positive marker and to our knowledge has not been shown to possess significant P-gp affinity. In addition to R123, permeability of P-gp substrates colchicine and digoxin were assessed in the optimized model. The differences in permeation rates for separate P-gp substrates can be attributed to the broad substrate affinities and capacities of the efflux transporters and their relative expression levels. Further studies can be performed to assess the effect of P-gp inhibition on the permeation of these substrates as well as inhibition of other efflux transporters such as BCRP as there is also fairly significant substrate overlap across several efflux transporter isoforms. Clozapine is an antipsychotic that has been shown to be highly metabolized and may potentially inhibit P-gp. Clozapine metabolites have also been demonstrated to have high BBB permeation, where additional studies using LC-mass spectrometry analysis and longer incubation time could be performed to elucidate the metabolic fate in the optimized triculture model (Hellman K, et al., ACS Chem Neurosci 2016; 7:668-80).

Although no metabolite peaks were observed in this study, a more rigorous separation analysis would be warranted to further investigate this possibility. Lastly, prazosin is a BCRP substrate that proved to have the lowest permeability of the selected markers. The low permeation of prazosin across the in vitro triculture model potentially suggests that functional BCRP activity is greater than that of P-gp or other efflux transporters, however further studies need to be performed to delineate the effects. The observed ranking of high and low BBB permeating compounds is ordered in a similar fashion to what has been seen by other both in vitro and in vivo. The observed permeability of a small library of compounds across the optimized direct contact triculture model suggests that it is a useful tool for further assessment of BBB permeation of new chemical entities as well understanding of the synergistic effects of direct cell-cell contacts.

Hence, we have established an enhanced physiologically relevant in vitro model of the BBB by culturing the astrocytes, pericytes, and HBEC-5i cells in a layered, direct contact manner resemblant of the in vivo NVU. We provide supporting evidence that the apical layering removes the physical barrier observed in conventional triculture models and supports the potential of synergistic interactions occurring to provide a phenotype closer to the NVU. In addition, to our knowledge we are one of the first laboratories to utilize a three stage multifactorial DOE based approach expedite optimization of a BBB in vitro model. Additional DOE based studies maybe performed to develop analogous models to mimic different pathologies of the brain, for example neurodevelopmental changes or neurodegenerative effects on the BBB with primary or proliferative cell lines.

To summarize, the Blood Brain Barrier in vitro screening approaches have traditionally focused on tightening the brain microvessel endothelium that line the capillaries, separate the blood from the neuronal environment, and maintain homeostasis. While screening models in the presence of astrocytes and pericytes in indirect contact to the BMECs have been developed, we postulated that direct contact of these cells, as found in vivo, would more adequately enhance in vitro-in vivo comparative studies. The direct layered culturing approach should enhance the synergistic effects by removing physical barriers and providing proximity so that secreted soluble factors and their effects on the regulation of the BMEC phenotype should be enhanced without added dilution and diffusion. Additionally, the ability for the model to rank established high and low brain permeating compounds eludes to its potential for BBB permeability screening of new chemical entities. This study also demonstrates the feasibility of using an informed DOE based approach to expedite culture development and can be further expanded for additional applications. Taken together, the direct contact triculture developed within appears to provide increased barrier properties that we theorize is attributable through facilitating adequate crosstalk between the three major cell types of the NVU. The findings of this work open the door for continued investigation of the roles of each NVU cell type and its influence on barrier properties, as well as the establishment of a fully human, physiologically relevant in vitro model that can be used for moderate throughput screening to rank order potential neuro therapeutic compounds. The following section is a practical application of this DOE methodology.

Astrocytes and other glial cells. Glial cells, or otherwise known as neuroglia, are found throughout the central nervous system (CNS) and neurovascular unit (NVU). These cells are responsible for secreting factors that maintain the CNS and the blood-brain barrier (BBB). Glial cells include astrocytes, microglia, and oligodendrocytes. The role of astrocytes in the BBB has been well established as a cell that secretes soluble factors that modulate the phenotype of the BBB (Abbott, Ronnback et al. Nature Reviews Neuroscience 2006, 7(1): 41-53). Though the presence and role of astrocytes is most predominant in the BBB compared to other glial cells, these additional cell types are known to play a role in BBB maintenance and development. For example, oligodendrocytes are known to secrete soluble factors that support BBB integrity, while microglia have been shown to become active in brain injury or trauma (Watzlawik, Warrington et al., Exp Rev Neurotherapeutics 2010, 10(3): 441-457). The use of astrocytes in the disclosed direct contact co- and triculture models would represent one state of the BBB. The addition of other glial cells, or the replacement of astrocytes with oligodendrocytes or microglial, in the direct contact models would be representative of another state of the BBB, e.g. brain injury, brain trauma, onset of neurodegenerative disease, etc.

BMECs vs. HBECs. Brain microvascular endothelial cells (BMECs) is the cell line most responsible for the formation of the blood-brain barrier (BBB) by forming restrictive tight junctions and expressing highly active efflux transports to prevent the permeation of xenobiotics into the brain. BMEC is most commonly used to refer to the endothelial cells that make up the BBB, there is a wide variety of endothelial cell lines that can be categorized as a type of BMEC. Human BMECs (HBEMCs) is a further classification of BMECs to those of human origin. Human brain endothelial cells (HBECs) is an additional way of classifying BMECs that are of human origin, and is often used interchangeable with HBMECs.

Types of BMECs. The breadth of BMECs use in in vitro models of the BBB is extensive. Cell lines can vary by species origin, proliferative state (primary cells taken from cadaver versus immortalized cell lines that have been transfected to express a phenotype through repeated culturing), cells derived from stem cells, and disease state (e.g. primary cells taken from cadaver patients having Alzheimer's or Parkinson's Disease). Helms et al. has extensively reviewed the various cell models that have been used for in vitro modeling of the BBB, which have included primary and immortalized cells from different species (e.g. murine, porcine, bovine, and human) and cells generated from human stem cells (Helms, H. C., et al., J. Cerebral Blood Flow Metabolism, 2016, 36(5): 862-890). The practice of isolating primary BMECs for their use in in vitro BBB models is well established for animal and human cell lines (Navone, S. E. et al., Nat Protoc 2013, 8(9): 1680-1693). By using this method, a person familiar with the field could isolate BMECs from various human cadaver sources to mimic a particular disease or age state of the BBB when used in the direct contact models. Table 5 below lists some examples of BMECs that are commonly used for in vitro BBB models and could be readily utilized in the direct contact model (Helms, H. C., et al., J. Cerebral Blood Flow Metabolism, 2016, 36(5): 862-890; Weksler, B. et al., Fluids Barriers CNS. 2013, 10:16).

An important way to quantifying and characterizing the permeability of a tight junction is to measure the transendothelial electrical resistance (TEER, Ωcm²). The value of TEER reflects the integrity of cellular barriers and its resistance to ion movement across cell layers, even though it is highly sensitive to temperature, medium composition, passage number, etc.

Statistics. The distribution of permeability coefficients across the brain has not been well studied; however, some studies involving other membranes suggest that permeability can be normally or log-normally distributed based on the compound (Frum, Y., et al., Eur. J. Pharm Biopharm. 2007, 67(2): 434-439; Khan, G. M., et al., Int. J. Phar. 2005, 303(1-2): 81-87). Therefore, the data presented here has been subjected to both parametric and non-parametric tests. Studies were compared using the Mann-Whitney test or the Kruskal-Wallis test with a Dunn's post-hoc test. Additionally, all studies were also subjected to a two-tailed unpaired student's t-test or one-way ANOVA with a Bonferroni post-hoc test. Studies with p-values less than 0.05 were considered to have significant differences.

Currently, the clinical translation of neurotherapeutics significantly lags behind the rapid increase in neurological disorders being seen worldwide, thus it is imperative that new methodologies are established to help facilitate the pharmaceutical development of these agents. Many have theorized that a majority of to the difficulties associated with translation of these agents arises because of the highly restrictive nature of the Blood Brain Barrier (BBB) in vivo where preclinical in vitro screens that do not provide physiological semblance and rigor for lead candidate selection and optimization. Compound design and selection has traditionally focused on selected physiochemical properties (e.g., MW<400, high lipophilicity, and poor solubility) that have been conventionally considered as favorable for the ability to traverse the BBB. In addition, a significant focus has also been placed on demonstrating lower affinity and capacity for efflux transporters like P-gp to increase parenchymal exposure. Often overlooked is the role of metabolism in the BBB or the potential that compounds possessing these physicochemical properties may also cross in excess and potentially elicit neurotoxic effects (Pardridge W M, NeuroRx 2005; 2:3-14). We have hypothesized that by developing an in vitro BBB model where neuroactivity could also be assessed would better evaluate risks in earlier stages that would aid in Go/No Go decision making.

Approaches to developing more physiologically relevant cell based models of the BBB, where the properties of the brain microvessel endothelial cells (BMECs) are emphasized has been the industrial standard to attempt mitigation of attritions rates. However, these efforts typically lead to in vitro models that are somewhat predictive, but lack in vivo relevancy in the configuration of cells in the model and are predicated on the fact that they are more readily amenable to higher throughput screening demands associated with large compound libraries (Bicker J., et al., Eur J Pharm Biopharm 2014; 87:409-32). Additionally, these models emphasize BBB permeability and do not incorporate neuroactivity, associated toxicity or induction of neuronal function, into the in vitro screening.

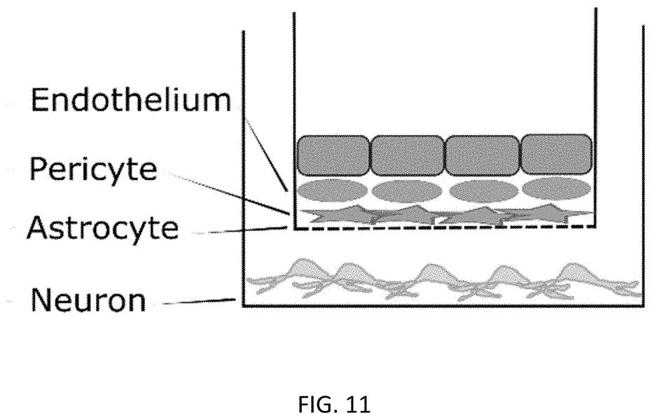

The study herein describes the development and early optimization of an in vitro NVU permeability-linked neuroactivity screening model. The model is predicated on utilizing the novel, direct contact BBB triculture for permeability assessment across human BMECs, pericytes, and astrocytes layered atop one another on a permeable filter support. The resulting flux of the compound then leads to exposure in the basal chamber, where human neuroblastoma cells (SH-SY5Y) that serve as a neuron surrogate are seeded and time dependent response can be evaluated, as depicted in FIG. 11. The SH-SY5Y cell line was utilized here due to their use in neurotoxicity studies, however it should be noted that this is proof of concept and the limitations of the SH-SY5Y cells are taken into account. To determine feasibility, BBB permeability linked neuroactivity was investigated utilizing marker compounds that were selected based upon reports indicating their effects on neuronal health and neurite outgrowth and the response to drug accumulation in the receiver chamber was determined. The model developed here encompasses the in vivo reality of an intended neurotherapeutic agent and its associated neuronal effects resulting in a physiologically relevant screening tool that may potentially be utilized to assess large libraries for hit and lead candidate selection.

Materials and Methods

Human brain astrocytes and vascular pericytes, astrocyte medium, pericyte medium, and astrocyte and pericyte growth factors were all obtained from ScienCell Research Laboratories (Carlsbad Calif., USA). HBEC-5i cells were purchased from ATCC (Manassas, Va., USA). SH-SY5Y neurons were graciously provided by Dr. Jean-Christophe Rochet (Purdue University, Department of Medicinal Chemistry and Molecular Pharmacology, West Lafayette, Ind., USA). TRANSWELL® filters of 12 mm 0.4 μm pore size, T75 culture flasks, MATRIGEL®, type I rat tail collagen, NUSERUM™, penicillin/streptomycin, and RPMI-1640 were purchased from Corning (Corning, N.Y., USA). Hank's balanced salt solution (HBSS) and Dulbecco's Modified Eagle Medium/Nutrient Mixture F-12 (DMEM/F-12) were obtained from Gibco (Carlsbad, Calif., USA). Fetal bovine serum (FBS), hydrocortisone, rhodamine 123 (R123), elacridar, carbamazepine, colchicine, clozapine, caffeine, melatonin, digoxin, cyclosporin A, and prazosin hydrochloride were purchased from MilliporeSigma (St. Louis, Mo., USA). Lapatinib was purchased from Attix Pharmaceuticals (Ontario, Canada). Radiolabeled [¹⁴C]-sucrose was obtained from Moravek Biochemicals Inc. (Brea, Calif., USA). HEPES (2-[4-(2-hydroxyethyl)piperazin-1-yl]ethanesulfonic acid) and calcium chloride dehydrate were obtained from J.T. Baker (Phillipsburg, N.J., USA). Dexamethasone and MTT (3-(4,5-dimethylthiazolyl-2)-2,5-diphenyl tetrazolium bromide) were obtained from MP Biomedicals (Santa Ana, Calif., USA). Endothelial cell growth supplement (ECGS) was purchased from Alfa Aesar (Haverhill, Mass., USA). Fluorescein isothiocyanate (FITC) labeled 4 kD and 40 kD dextrans were purchased from Chondrex (Redmond, Wash., USA). Fluorescein isothiocyanate (FITC) labeled 10 kD dextran was purchased from TCI America (Portland, Oreg., USA). Poly-L-lysine (PLL) was purchased from Trevigen (Gaithersburg, Md., USA). Neurite Outgrowth Staining Kit was purchased from Molecular Probes (Eugene, Oreg., USA).

Cell Culture. Human Brain Endothelial Cells (HBEC-5i) were maintained in T-75 culture flasks pre-coated with Type I rat tail collagen with medium changes every 3 days and subculturing at 80-90% confluency—cells were utilized between passage 22 and 30. HBEC-5i culture medium was made up of Dulbecco's Modified Eagle Medium/Nutrient Mixture F-12 (DMEM/F-12) supplemented with 10% FBS, 15 mM HEPES, and 40 ug/mL endothelial cell growth supplement (ECGS). Human astrocytes and human brain vascular pericytes are maintained in T-75 culture flasks pre-coated with poly-L-lysine with medium changes every 3 days and passaging at 80-90% confluency, where the cells were utilized between passages 4 and 10. Astrocyte culture medium was made up of Astrocyte Medium supplemented with 5% FBS, astrocyte growth supplement, and penicillin/streptomycin. Pericyte culture medium was comprised of Pericyte Medium supplemented with 5% FBS, pericyte growth supplement, and penicillin/streptomycin. Human neuroblastoma cell line, SH-SY5Y, was maintained in T-75 culture flasks with medium changes every 3 days and passaging at 80-90% confluency. SH-SY5Y cells were grown in RPMI 1640 with L-glutamine and 25 mM HEPES supplemented with 10% NUSERUM™ and 1% penicillin/streptomycin. SH-SY5Y cells were used between passage 10 and 17 in all experiments.

Optimizing Plating of Neurons with Direct Contact Triculture

Seeding density and time of introduction of with the direct contact triculture with the SH-SY5Y neurons was optimized using a full factorial design of the two factors (Table 5). Plating methods of the BBB triculture is explained in the following section. Neurons were plated in a separate 12-well plate at 25,000, 50,000, or 75,000 cells/cm² 24 hours prior to placing the direct contact triculture atop the neurons. Neurons were cultured with the apical triculture starting at 3 or 7 days post endothelial cells plating, and cultured until day 9 post endothelium plating. Cultures were maintained at 37° C. and 5% CO₂ with complete endothelial medium in the apical chamber and neuronal medium in the basolateral chamber with medium changes every other day. Optimized conditions were selected based on paracellular permeability of a 4 kD fluorescein labeled dextran.

Plating Direct Contact Triculture with Neurons in the Basolateral Chamber Seeding of the direct contact triculture was done following the optimized procedure. Briefly, 12mm, 0.4 μm pore polyester TRANSWELL® filters were pre-coated with 5 μg/cm² poly-L-lysine. Astrocytes were seeded at a density of 20,000 cells/cm² and allowed to grow for 48 hours prior to seeding pericytes atop the astrocyte cell layer at a density of 20,000 cells/cm². After 48 hours of pericyte growth, MATRIGEL® at a density of 25 μL/cm² in HBSS was added to the astrocyte-pericyte lawn and allowed to incubate at 37° C. for 45 minutes. MATRIGEL® was removed and HBEC-5i cells were seeded directly atop the astrocyte-pericyte ECM coated lawn at a density of 80,000 cells/cm² and maintained. SH-SY5Y cells were introduced to the direct contact triculture 3 days post endothelial cell plating. Neurons were seeded 24 hours prior to incorporation in a 12-well plate at a density of 25,000 cells/cm². Filter supports containing the direct contact triculture were placed above the culture neurons in the basolateral chamber of the well plate. Cultures were maintained with complete endothelial medium on the apical side of the filter and complete neuronal medium in the basolateral chamber. Cultures were utilized for assessing permeability and neuroactivity screening at 9 days post endothelial cell seeding.

TABLE 5
Optimization of the NVU Model

| | | Incorpo- | % Papp (4 kD FITC- |
|---|---|---|---|
| | SH-SY5Y Density | ration | Dextran) change of |
| Condition | (×10³ cells/cm²) | Day* | control |
| 1 | 25 | 3 | −11% |
| 2 | 25 | 7 | +2% |
| 3 | 50 | 3 | −16% |
| 4 | 50 | 7 | +6% |
| 5 | 75 | 3 | −3% |
| 6 | 75 | 7 | −4% |
| Optimized | — | — | — |
| Triculture | | | |
| Control⁺ | | | |

*day post endothelial cell plating
⁺all conditions performed at n = 1, control at n = 2

Permeability Assays

Prior to commencing all assays, cells were washed (2×) with PBS to remove residual medium and then left to incubate in HBSS for 30 minutes at 37° C. to equilibrate. Apparent permeability of paracellular markers 4 kD, 10 kD, and 40 kD FITC-dextran, and [¹⁴C]-sucrose was performed at 37° C. on a rocking platform with samples pulled at 15, 30, 45, 60, and 90 minutes, where all studies were conducted under sink conditions. Dextran solutions and [¹⁴C]-sucrose were prepared at initial concentrations of 250m/mL and 0.25 μCi respectively in HBSS containing 0.50% DMSO. Dextran solutions were analyzed using a BioTek Synergy 4 plate reader with excitation at 485 nm and emission at 530 nm while [¹⁴C]-sucrose was assessed via scintigraphy.

The effective permeability coefficients of selected markers (caffeine, carbamazepine, melatonin, clozapine, digoxin, cyclosporine A, lapatinib, and prazosin) was performed at initial concentrations of 25 or 50 μM in HBSS containing 0.50% DMSO from 10 mM concentrated stock solutions in DMSO for each compound. Samples were removed at 30, 60, 90, 120, 150, and 180 minutes for determining permeation rates and the remaining neurons then washed for the evaluation of neuroactivity. All samples for these compounds were analyzed using high performance liquid chromatography (HPLC). Apparent permeability (Pₐₚₚ) and flux (J) were calculated using the following equation (eq. 3):

$${C}_{0} \cdot {P}_{apparent} = \frac{dM / dT}{A} = Flux \qquad ( eq . 3 )$$

wherein dM/dT is the amount of material that moves across the filter over time, Co is the initial concentration in the donor (apical) chamber, and A is the surface area of the filter support.

Apparent permeability of P-glycoprotein (P-gp) substrate rhodamine 123 (R123) was measured in the presence and absence of elacridar, a P-gp inhibitor. Working solutions of R123 at 10 μM and elacridar at 2 μM were prepared in HBSS with 1% DMSO. Replicates in the presence of inhibitor were incubated with elacridar for 45 minutes prior to the start of R123 permeation, while samples without inhibitor were incubated in blank HBSS. Samples were collected at 30, 60, 90, and 120 minute intervals and assessed using a BioTek Synergy 4 plate reader at excitation of 485 nm and emission of 530 nm. Apparent permeability was measured using eq. 3 shown above.

High Performance Liquid Chromatography

All compounds were analyzed using an Agilent 1100 reversed phase HPLC equipped with a variable wavelength detector (VWD). All methods were run isochratically using water and acetonitrile (ACN) through an ASCENTIS® C-18 15×4.6 mm, 5 μm column at 25 μL injections. Caffeine mobile phase consisted of 90:10, water:ACN, flow rate of 1.0 mL/min run at ambient temperature and analysis at 275 nm. Carbamazepine was run with 65:35, water:ACN at 1.5 mL/min with a column temperature of 40° C. and observed at 284 nm. Clozapine utilized a mobile phase of 45:55, water:ACN at 1.5 mL/min flow rate and 40° C. column temperature with analysis at 254 nm. Colchicine analysis was performed using a mobile phase of 75:25, water:ACN at a flow rate of 1.5 mL/min, 40° C. column temperature, and wavelength of 354 nm. Cyclosporin A was run using a mobile phase of 30:70, water:ACN at 1.5 mL/min flow rate, 40° C. column temperature, and measured at 214 nm. Digoxin utilized a mobile phase of 30:70, water:ACN at a flow rate of 1.1 mL/min, 40° C. column temperature, and observed at 218 nm. Lapatinib utilized a mobile phase of 40:60, water:ACN at a flow rate of 1.0 mL/min, a column temperature of 25° C., and VWD detection at 232 nm. Melatonin was measured using a mobile phase of 75:25, water:ACN with a flow rate of 1.5 mL/min, 40° C. column temperature, and measured at 222 nm. Prazosin was analyzed with a mobile phase of 65:35, water:ACN at a flow rate of 1.5 mL/min, a 40° C. column, and wavelength of 254 nm.

Neuroactivity Assessment

Neuroactivity was assessed after 3 hour BBB permeability of marker compounds caffeine, carbamazepine, clozapine, digoxin, prazosin, and cyclosporin A using the dual fluorescent dye MOLECULAR PROBES® Neurite Outgrowth Staining Kit which provides neuronal viability and degree of neurite outgrowth differences in comparison to a control. Following incubation, drug was removed from the neuronal cells and neurons were washed with fresh HBSS. Staining solution containing cell viability indicator and cell membrane stain was prepared according to manufacturer recommendations in fresh HBSS and added to neuron samples. After cells were incubated with the stain for 20 minutes at room temperature the stain was removed, cells were gently washed with fresh HBSS, and background suppression solution was added for analysis. Fluorescence quantification was measured using a BioTek Neo2 plate reader where the viability stain was measured at excitation and emission of 483 nm and 525 nm, whereas the cell membrane stain was measured at excitation and emission wavelength of 535 nm and 590 nm. All samples were compared to a control containing vehicle alone (0.50% DMSO in HBSS) and cell free controls were used to account for background fluorescence. Qualitative images were obtained using the BioTek Cytation 3 with the 20× objective for bright field and fluorescent pictures. Green Fluorescent Protein (GFP) and Texas Red filters were used for to observe fluorescence in each sample.

Triculture Cell Viability Assay

The viability of the direct contact triculture at the completion of the neuroactivity measurements was inferred from the mitochondrial oxidation of 3-(4,5-dimethylthiazolyl-2)-2,5-diphenyl tetrazolium bromide (MTT) dye. At the end of BBB permeability linked neuroactivity studies triculture plated filters were moved to a blank chamber well and the apical solution was removed from the filter support then washed with fresh HBSS. The triculture was incubated with 450 μL of fresh HBSS and 50 μL of 5 mg/mL MTT stock solution in HBSS for 4 hours at 37° C., blank HBSS was kept in the basolateral chamber to ensure cells were not directly exposed to air. After incubation, MTT solution was removed and replaced with 300 μL of DMSO to lyse cells and solubilize the mitochondria-generated formazan salt. A sample volume of 50 μL was diluted an equal volume of fresh DMSO in a 96-well plate. Absorbance was measured at 560 nm using a BioTek Powerwave HT plate reader. Samples were compared to a control of triculture incubated with vehicle alone (0.50% DMSO in HBSS) over the course of permeability measurement.

Results

NVU Optimization

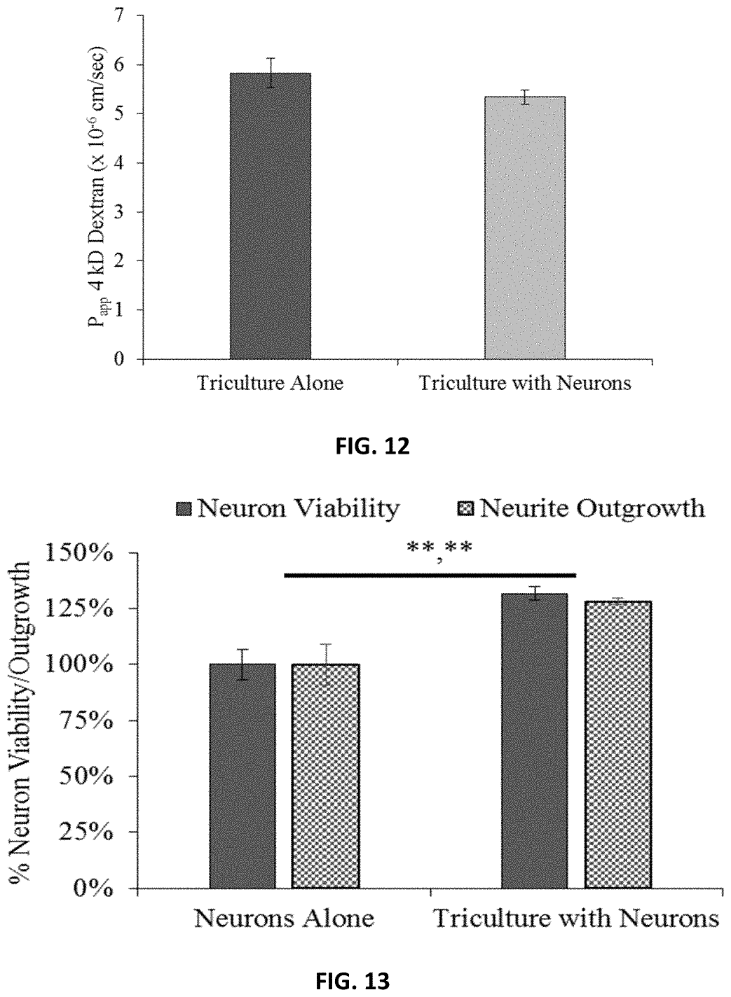

A full factorial design was used to determine the optimal conditions using three levels of neuron density and two times of inclusion. Upon analysis, the seeding density and time of inclusion of the SH-SY5Y neurons with the DOE optimized direct contact triculture were determined to be the two most important factors that influence the barrier restrictiveness of the NVU model. Neuronal influence on BBB properties was assessed based on the change in paracellular permeability marker compound, 4 kD FITC-dextran. Results revealed that, regardless of cell density, introducing neurons to the basal chamber at day 7 post HBEC-5i plating had a lesser impact on barrier properties in contrast to introduction earlier in culture at day 3. Introducing neurons on day 3 at 25,000 and 50,000 cells/cm² resulted in the largest decreases in BBB permeation, 11% and 16% decreases respectively (Table 5). A neuron density of 25,000 cells/cm² introduced at day 3 post endothelial cell plating were the conditions chosen for all further studies to facilitate neuroactivity measurements. The selected optimized conditions were repeated to confirm the decrease in the permeation rate for 4 kD FITC-dextran with the addition of neurons in the basolateral chamber. The Pₐₚₚ observed for the triculture alone was 5.83±0.30×10⁻⁶ cm/sec compared to 5.34±0.15×10⁻⁶ cm/sec with neurons in the basolateral chamber resulting in an apparent decrease of 9% in permeability (p>0.05) (FIG. 12).

Neuron Viability and NVU Marker Compounds

Viability and outgrowth of neurons throughout the length of coculture in the presence of the direct contact triculture was assessed. SH-SY5Y neuronal cells were cultured alone in a well plate for 8 days or for 24 hours alone then combined with the apical triculture for an additional 7 days. Viability and outgrowth of neurons cultured alone was normalized to the control values of 100±7% and 9% respectively. In the presence of the triculture neuron viability increased by 32±3% and outgrowth increased by 28±1% in comparison to the neurons alone (FIG. 13). This was determined based on quantifying relative fluorescence intensity of the neuron viability and neurite outgrowth stain using the Molecular Probes® Neurite Outgrowth Staining Kit.

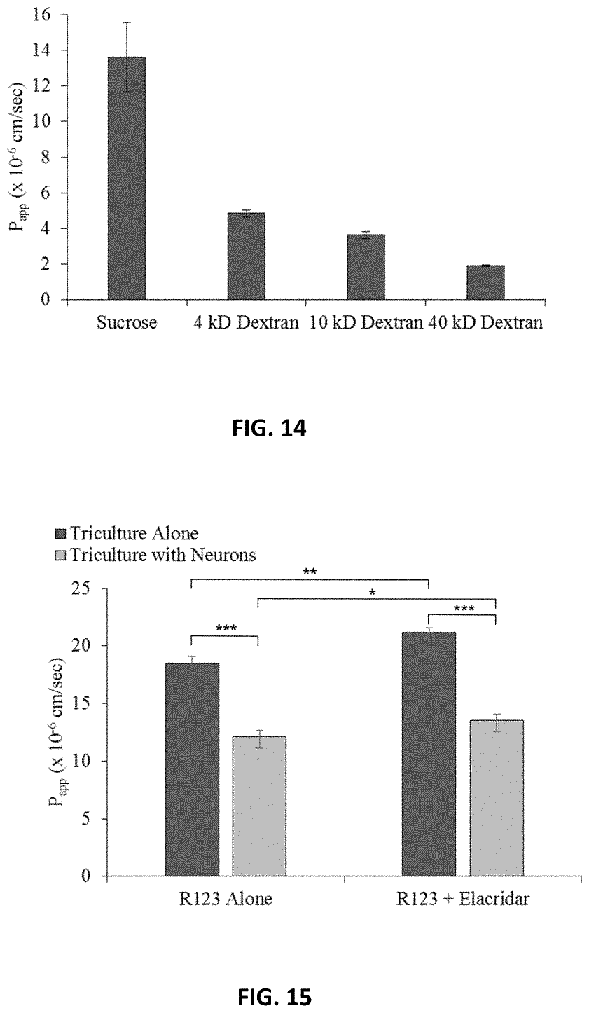

Four paracellular markers of increasing hydrodynamic radii were used to assess the tightness of the NVU model. Apparent permeability of [¹⁴C]-sucrose (4.6 Å) was 13.61±1.94×10⁻⁶ cm/sec, followed by 4 kD FITC-dextran (14 Å) at 4.85±0.20×10⁻⁶ cm/sec, 10 kD FITC-dextran (23 Å) at 3.64±0.20×10⁻⁶ cm/sec, and 40 kD FITC-dextran (45 Å) at 1.92±0.05×10⁻⁶ cm/sec (FIG. 14).

The function of efflux transporter P-glycoprotein (P-gp) is also a key validation characteristic of any in vitro BBB model. P-gp function was assessed in the NVU model using P-gp substrate rhodamine (R123) in the presence and absence of P-gp inhibitor elacridar (FIG. 15). In the NVU model, the Pₐₚₚ of R123 alone was 12.12±0.57×10⁻⁶ cm/sec versus 13.56±0.50×10⁻⁶ cm/sec in the presence of elacridar (p<0.05). In comparison, the Pₐₚₚ of R123 alone and in the absence of elacridar across the BBB direct contact triculture alone was 18.52±0.58×10⁻⁶ cm/sec and 21.14±0.46×10⁻⁶ cm/sec respectively (p<0.01). The triculture alone shows a greater increase in the Pₐₚₚ of R123 in the presence of inhibitor (14% increase) compared to the NVU model (12% increase), however the overall permeability of R123 with and without inhibitor is decreased in the NVU model compared to the triculture alone (p<0.001).

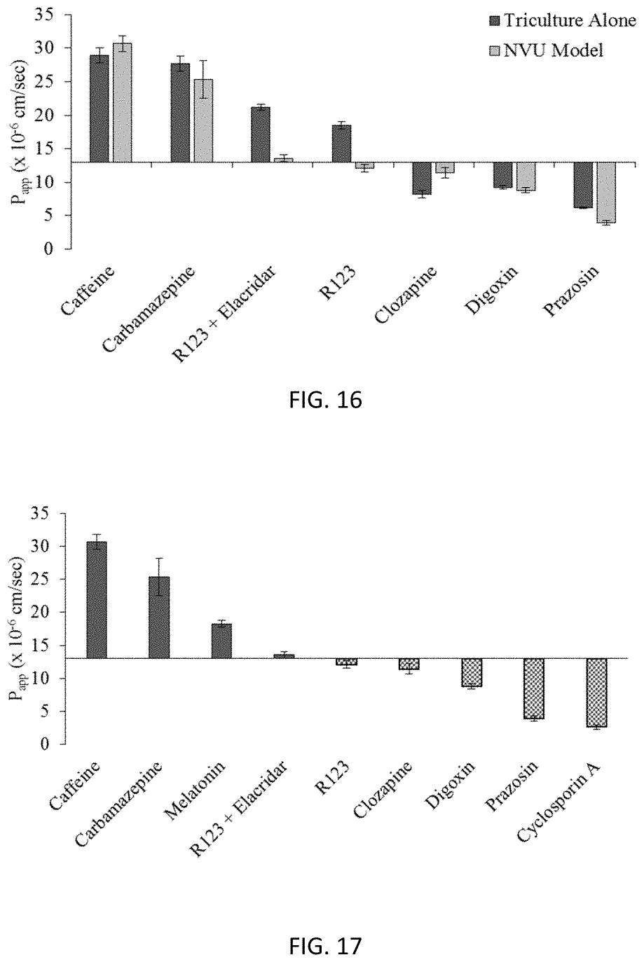

The apparent permeability across the NVU model was measured for a number of BBB high and low permeating compounds to validate the screening tool for use in ranking potential therapeutic agents based on permeation rates (FIG. 16). Caffeine (Pₐₚₚ=30.70±1.18×10⁻⁶ cm/sec), carbamazepine (Pₐₚₚ=25.37±2.80×0⁻⁶ cm/sec), melatonin (Pₐₚₚ=18.29±0.50×10⁻⁶ cm/sec), and R123 in the presence of elacridar (Pₐₚₚ=13.56±0.50×10⁻⁶ cm/sec) are positive BBB permeants. R123 alone (Pₐₚₚ=12.12±0.57×10⁻⁶ cm/sec), clozapine (Pₐₚₚ=11.44±0.78×10⁻⁶ cm/sec), digoxin (Pₐₚₚ=8.78±0.37×10⁻⁶ cm/sec), prazosin (Pₐₚₚ=3.90±0.35×10⁻⁶ cm/sec), and cyclosporine A (Pₐₚₚ=2.61±0.37×10⁻⁶ cm/sec) are BBB negative permeants. Lapatinib was also tested for permeability, but was undetectable in the receiver chamber after 3 hours. The BBB permeants that were tested across both the optimized triculture without neurons and the NVU model were plotted for comparison. Of the markers screened, significant decreases in permeation rates across the NVU model were observed for R123 with elacridar (p<0.001), R123 alone (p<0.001), and prazosin (p<0.01) while a significant increase in permeation was seen for clozapine (p<0.01) all in comparison to the optimized triculture alone (FIG. 17).

BBB Linked Neuroactivity

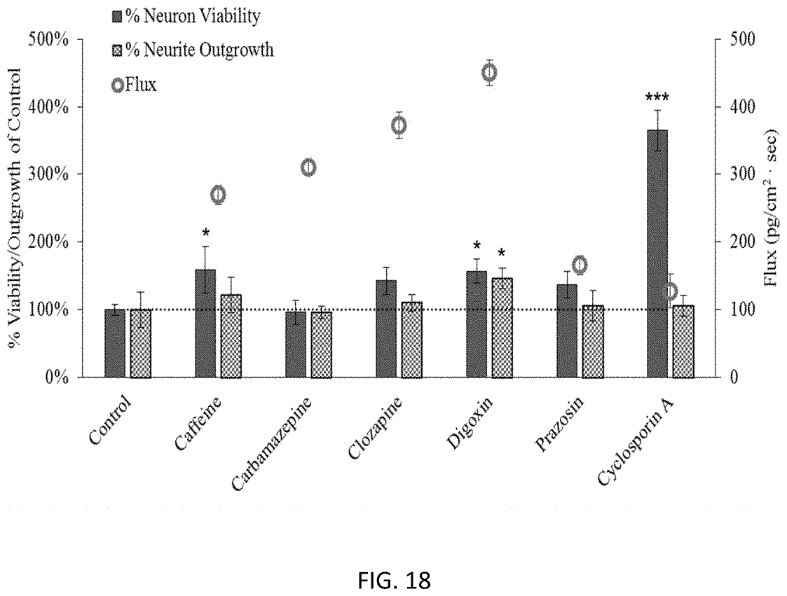

Relative neuroactivity was measured following 3 hour BBB permeability at an initial concentration of 50 μM in the NVU model where drug accumulated in the receiver chamber containing SH-SY5Y neuronal cells over the course of permeation across the apical direct contact triculture (FIG. 18). This study was performed at n=4 and neuroactivity (neuronal viability and outgrowth) data is reported as a percent of control NVU SH-SY5Y cells with vehicle (0.50% DMSO) alone, and flux reported for each compound to represent the amount accumulated in the neuronal chamber. In comparison to the control (neuron viability was 100±8%, where neurite outgrowth was 100±26%) caffeine accumulation resulted in a significant increase in viability with a non-significant increase in outgrowth (159±34%, p<0.05; 122±26%; flux=269±14 pg/cm²·sec). Carbamazepine (96±18%; 96±9%; flux=310±9 pg/cm²·sec) demonstrated negligible changes in viability and outgrowth while clozapine (143±20%; 111±12%; flux=372±19 pg/cm²·sec) and prazosin (137±19%; 106±22%; flux=166±14 pg/cm²·sec) demonstrated insignificant increases in both viability and outgrowth in comparison to the control. Permeability linked neuroactivity of the SH-SY5Y cells in response to digoxin accumulation resulted in significant increases in both viability and outgrowth when compared to the control neuronal cells (157±18%, p<0.05; 147±15%, p<0.05; flux=450±19 pg/cm²·sec). Lastly, cyclosporin A accumulation resulted in the largest increase in neuronal viability and insignificant changes in neurite outgrowth (365±30%, p<0.001; 106±15%; flux=127±27 pg/cm²·sec).

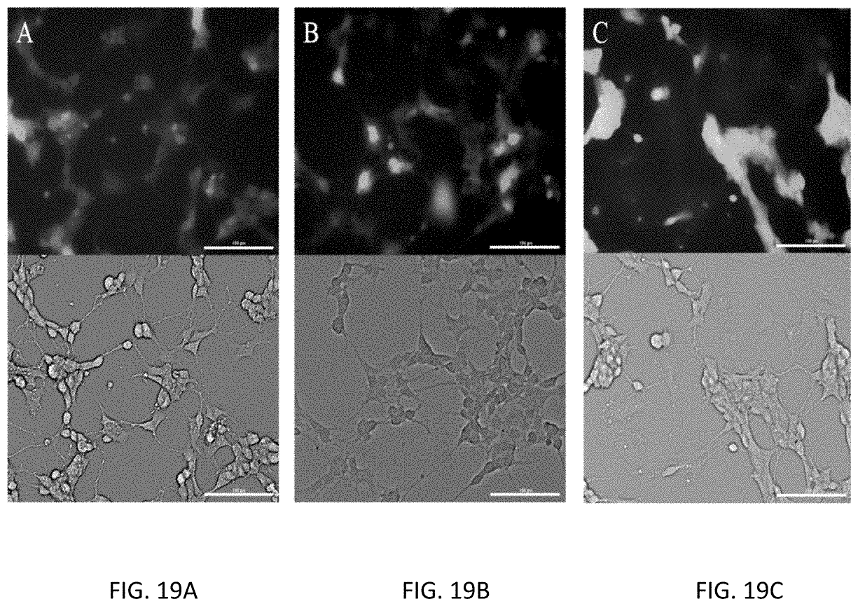

Representative images of neuronal viability and outgrowth staining are presented for the control, digoxin, and cyclosporin A samples (FIG. 19). In comparison to the control (panel A), punctate neurite projections are observable in the fluorescent (red) and bright field images of digoxin SH-SY5Y neurons (panel B), while a qualitative intensified green fluorescence and diffuse outgrowth is observed for cyclosporin A neuronal cells (panel C). These qualitative observations correlate to the quantifiable data obtained from the relative fluorescence results (FIG. 18).

BBB Triculture Viability

Viability of the apical triculture cells was inferred using an MTT assay to determine if observed neuronal effects or flux were due to changes in the integrity of the triculture cells. Inferred viability of the triculture cells is normalized to the triculture cells of the control NVU and reported as percent of the control. The viability of the triculture following 3 hours of caffeine (104±25%), carbamazepine (103±5%), clozapine (99±4%), digoxin (94±5%), prazosin (82±6%), and cyclosporin A (103±2%) permeation resulted in no significant changes in comparison to the triculture of the control NVU (100±2%, p>0.05).

To summarize, we have demonstrated that the presence of a BBB model in neuroactivity screening is essential for adequately mimicking the path of a compound in vivo. The BBB triculture presents not only as a permeation but also as a metabolic barrier of entry into the brain parenchyma or neuronal chamber of the in vitro model. In vivo the NVU responds to neuronal demands based on synergistic signaling from the neurons through direct contacts with astrocytes via neuronal secretion of a number of soluble factors. This in turn modulates vascular diameter by pericyte action to protect the parenchyma (Hamilton N B, et al., Front Neuroenergetics 2010, 2:5). We have demonstrated, in the optimized NVU model, that there is synergistic signaling occurring through all cell types by soluble factor secretion.

Furthermore, we have developed a physiologically relevant cell-based model of the NVU that incorporates BBB permeation and linked neuroactivity into a single screening tool. Additionally, we have demonstrated that the incorporation of all four cell types of the NVU leads to increased phenotypic expression of the BBB as well as cellular viability of the neuronal cells. The utility of the model serves to mimic the in vivo situation a therapeutic agent may encounter when attempting to cross the BBB into the brain parenchyma. By implementing this screening tool in pharmaceutical development of neurotherapeutic agents, as well as other classes of drugs, there is potential to decrease the resources needed for ranking hit and lead candidate compounds through the evaluation of BBB permeability linked neuroactivity using a single in vitro screening tool.

While various embodiments of Blood Brain Barrier models and methods to generate and use the same have been described in considerable detail herein, the embodiments are merely offered as non-limiting examples of the disclosure described herein. It will therefore be understood that various changes and modifications may be made, and equivalents may be substituted for elements thereof, without departing from the scope of the present disclosure. The present disclosure is not intended to be exhaustive or limiting with respect to the content thereof.

Further, in describing representative embodiments, the present disclosure may have presented a method and/or a process as a particular sequence of steps. However, to the extent that the method or process does not rely on the particular order of steps set forth therein, the method or process should not be limited to the particular sequence of steps described, as other sequences of steps may be possible. Therefore, the particular order of the steps disclosed herein should not be construed as limitations of the present disclosure. In addition, disclosure directed to a method and/or process should not be limited to the performance of their steps in the order written. Such sequences may be varied and still remain within the scope of the present disclosure.

We claim:

1. A method for preparing a multi-cellular neurovascular unit (NUV) for Blood Brain Barrier (BBB) linked neuroactivity evaluation comprising the steps of:
a) preparing a cell culture plate with a first support membrane;
b) seeding a first cell line on said first support membrane and proliferating said first cell line;
c) seeding a second cell line over said first cell line and proliferating said second cell line;
d) coating the lawn of said first and second cell lines with an extracellular matrix (ECM);
e) seeding a third cell line over ECM coated proliferated cell lawn of said first and second cell line and proliferating said third cell line together with said first and second cell lines to afford a triculture system;
f) seeding a neuron cell line or a surrogate on a second support membrane; and
g) placing said first support membrane comprising said triculture system above said neuron cell line or a surrogate into a cell culture chamber to afford an in vitro neurovascular unit (NVU).

2. The method of claim 1, wherein said first cell line is astrocytes or other glial cells and said second cell line is pericytes.

3. The method of claim 2, wherein said first cell line of astrocytes or other glial cells has a seeding density of about 20,000 cells/cm² and said second cell line of pericytes has a seeding density of about 20,000 cells/cm², optimizable using the method Design of Experiments (DOE).

4. The method of claim 1, wherein said third cell line is proliferative human derived cerebral microvessel endothelial cells (HBEC-5i).

5. The method of claim 1, wherein said third cell line is preprogrammed induced pluripotent stem cells.

6. The method of claim 1, wherein said third cell line comprises brain microvessel endothelial cells (BMECs) of human or animal origin, primary, immortalized, normal or in a diseased state, or Human Brain Endothelial Cells (HBECs).

7. The method of claim 6, wherein said third cell line of brain microvessel endothelial cells (BMECs) of human or animal origin, primary, immortalized, normal or in a diseased state, or Human Brain Endothelial Cells (HBECs) has a seeding density of about 80,000 cells/cm², optimizable using the method Design of Experiments (DOE).

8. The method of claim 1, wherein said neuron cells or a surrogate neuronal cells comprises human neuroblastoma cell lines, preprogrammed induced pluripotent stem cells of differing phenotypes, isolated primary human or animal neurons that are obtained from different brain sections, and human or animal neuronal cell lines.

9. The method of claim 8, wherein said a neuronal cell line has a seeding density of from about 5,000 to about 500,000 cells/cm², optimizable using the method Design of Experiments (DOE).

10. The method of claim 1, wherein performance of said NVU cell culture system is optimal when proliferated cell lines reach confluency.

11. A multi-cellular cell culture system of in vitro neurovascular unit (NVU) for Blood Brain Barrier (BBB) linked neuroactivity evaluation comprising:
a) a cell culture plate with a first support membrane;
b) a triculture system comprising a first cell line, a second cell line and a third cell line, wherein said first cell line and second cell line are seeded on said first support membrane and proliferating before coating the lawn of said first and second cell lines with an extracellular matrix (ECM) and then seeding said third cell line to enable BBB formation;
c) a neuron cell line or a surrogate, wherein said neuron cell line or surrogate is seeded on a second support membrane prior to placing said triculture system atop said neuron cell line or a surrogate to afford an in vitro neurovascular unit (NVU); and
d) said NVU is maintained with complete endothelial medium on the apical side of the filter and complete neuronal medium in the basolateral chamber until being demonstrated as optimal for permeability and neuroactivity assays.

12. The cell culture system according to claim 11, wherein said first cell line is astrocytes or other glial cells and said second cell line is pericytes.

13. The cell culture system according to claim 12, wherein said first cell line of astrocytes or other glial cells has a seeding density of about 20,000 cells/cm² and said second cell line of pericytes has a seeding density of about 20,000 cells/cm², optimizable using the method Design of Experiments (DOE).

14. The cell culture system according to claim 11, wherein said third cell line is proliferative human derived cerebral microvessel endothelial cells hCMEC/D3 or preprogrammed induced pluripotent stem cells.

15. The cell culture system according to claim 11, wherein said third cell line is brain microvessel endothelial cells (BMECs) of human or animal origin, primary, immortalized, normal or in a diseased state, or Human Brain Endothelial Cells (HBECs).

16. The cell culture system according to claim 15, wherein said third cell line of brain microvessel endothelial cells (BMECs) of human or animal origin, primary, immortalized, normal or in a diseased state, derived from induced pluripotent stem cells,or a surrogate of Brain Endothelial Cells (HBECs) has a seeding density of about 80,000 cells/cm², optimizable using the method Design of Experiments (DOE).

17. The cell culture system according to claim 11, wherein said neuron cell or surrogate comprises human neuroblastoma cell lines, preprogrammed induced pluripotent stem cells of differing phenotypes, isolated primary human or animal consisting of both healthy or diseased state neurons that are obtained from different brain sections, and human or animal neuronal cell lines.

18. The cell culture system according to claim 17, wherein said human neuroblastoma cell line has a seeding density of about 5,000 to about 500,000 cells/cm², optimizable using the method Design of Experiments (DOE).

19. The cell culture system according to claim 11, wherein performance of said NVU cell culture system is at its best when proliferated cells reach confluency.

20. A cell culture kit of multi-cellular cell culture system of in vitro neurovascular unit (NVU) for Blood Brain Barrier (BBB) linked neuroactivity evaluation comprising:
a) a cell culture plate with a first support membrane;
b) a triculture system comprising a first cell line, a second cell line and a third cell line, wherein said first cell line and second cell line are seeded on said first support membrane and proliferating before coating the lawn of said first and second cell lines with an extracellular matrix (ECM) and then seeding said third cell line to enable BBB formation;
c) a neuron cell line or a surrogate, wherein said neuron cell line or surrogate is seeded on a second support membrane prior to placing said triculture system atop said neuron cell line or a surrogate to afford an in vitro neurovascular unit (NVU);
d) said NVU is maintained with complete endothelial medium on the apical side of the filter and complete neuronal medium in the basolateral chamber until being demonstrated as optimal for permeability and neuroactivity assays; and
e) supporting accessories.
